SEQUENCE LISTING (24 sequences)

SEQ ID NO: 1 - DNA, 423 bp, Escherichia coli
  source 1..423: mol_type = genomic DNA; note = fucose mutarotase(fucU)
atgctgaaaacaatttcgccgttaatttctcccgaactattgaaagtgctggcagagatgggacatggagatgaaattattttttccgatgctcactttcccgcccattcgatgggaccgcaggtgatccgcgctgatggcctgttggtgagcgacttgctccaggcgattatcccgttatttgaactggacagttatgcaccgccgctggtgatgatggcggcggtagaaggtgacactctcgatcctgaagtagaacgacgttaccgtaatgcgctttcactacaagccccgtgtcctgacatcatccgcatcaatcgttttgcgttttatgaacgggcgcaaaaagcctttgcgatcgttatcacaggcgaacgagcgaagtacgggaatattcttttaaaaaaaggggtaacaccgtaa

SEQ ID NO: 2 - DNA, 1776 bp, Escherichia coli
  source 1..1776: mol_type = genomic DNA; note = fucose isomerase(fucI)
atgaaaaaaatcagcttaccgaaaattggtatccgcccggttattgacggtcgtcgcatgggtgttcgtgagtcgcttgaagaacaaacaatgaatatggcgaaagctacggccgcactgctgaccgagaaactgcgccatgcctgcggagctgccgtcgagtgtgtcatttccgatacctgtatcgcgggtatggctgaagccgctgcttgcgaagaaaaattcagcagtcagaatgtaggcctcaccattacggtaacgccttgctggtgctatggcagtgaaaccatcgacatggatccaacccgcccgaaggccatttggggctttaacggcactgaacgccccggcgctgtttacctggcagcggctctggcagctcacagccagaaaggcatcccagcattctccatttacggtcatgacgttcaggatgccgatgacacatcgattcctgccgatgttgaagaaaaactgctgcgctttgcccgcgccggtttggccgtcgccagcatgaaaggtaaaagctatctgtcgctgggcggcgtttcgatgggtatcgccggttccattgttgatcacaacttctttgaatcctggctgggaatgaaagtccaggcggtggatatgaccgaactgcgtcgccgtatcgatcagaagatttacgacgaagccgaattggaaatggcactggcctgggctgataaaaacttccgctatggcgaagatgaaaataacaaacagtatcaacgtaatgccgagcaaagccgcgcagttctgcgcgaaagtttactgatggcgatgtgtatccgcgacatgatgcaaggcaacagcaaactggccgatattggtcgcgtggaagaatcacttggctacaacgccatcgctgcgggcttccaggggcaacgtcactggaccgatcaatatcccaatggtgacaccgccgaagcgatcctcaacagttcatttgactggaatggcgtgcgcgaaccctttgtcgtggcgaccgaaaacgacagtcttaacggcgtggcaatgctaatgggtcaccagctcaccggcaccgctcaggtatttgccgatgtgcgtacctactggtcaccagaagcaattgagcgtgtaacggggcataaactggatggactggcagaacacggcatcatccatttgatcaactccggttctgctgcgctggacggttcctgtaaacaacgcgacagcgaaggtaacccgacgatgaagccacactgggaaatctctcagcaagaggctgacgcttgcctcgccgctaccgaatggtgcccggcgatccacgaatacttccgtggcggcggttactcttcccgcttccttaccgaaggcggcgtcccgttcaccatgactcgtgtcaacatcatcaaaggcctgggaccggtactgcaaatcgcggaaggctggagcgtggaattgccgaaggatgtgcatgacatcctcaacaaacgcaccaactcaacctggccaaccacctggtttgcaccgcgcctcaccggtaaagggccgtttacggatgtgtactcggtaatggcgaactggggcgctaaccatggggttctgaccatcggccacgttggcgcagactttatcactctcgcctccatgctgcgtatcccggtatgtatgcacaacgttgaagagaccaaagtgtatcgtccttctgcctgggctgcgcacggcatggatattgaaggccaggattaccgcgcttgccagaactacggtccgttgtacaagcgttaa

SEQ ID NO: 3 - DNA, 1419 bp, Escherichia coli
  source 1..1419: mol_type = genomic DNA; note = fuculose kinase(fucK)
atgaaacaagaagttatcctggtactcgactgtggcgcgaccaatgtcagggccatcgcggttaatcggcagggcaaaattgttgcccgcgcctcaacgcctaatgccagcgatatcgcgatggaaaacaacacctggcaccagtggtctttagacgccattttgcaacgctttgctgattgctgtcggcaaatcaatagtgaactgactgaatgccacatccgcggtatcgccgtcaccacctttggtgtggatggcgctctggtagataagcaaggcaatctgctctatccgattattagctggaaatgtccgcgaacagcagcggttatggacaatattgaacggttaatctccgcacagcggttgcaggctatttctggcgtcggagcctttagtttcaatacgttatataagttggtgtggttgaaagaaaatcatccacaactgctggaacgcgcgcacgcctggctctttatttcgtcgctgattaaccaccgtttaaccggcgaattcactactgatatcacgatggccggaaccagccagatgctggatatccagcaacgcgatttcagtccgcaaattttacaagccaccggtattccacgccgactcttccctcgtctggtggaagcgggtgaacagattggtacgctacagaacagcgccgcagcaatgctcggcttacccgttggcataccggtgatttccgcaggtcacgatacccagttcgccctttttggcgctggtgctgaacaaaatgaacccgtgctctcttccggtacatgggaaattttaatggttcgcagcgcccaggttgatacttcgctgttaagtcagtacgccggttccacctgcgaactggatagccaggcagggttgtataacccaggtatgcaatggctggcatccggcgtgctggaatgggtgagaaaactgttctggacggctgaaacaccctggcaaatgttgattgaagaagctcgtctgatcgcgcctggcgcggatggcgtaaaaatgcagtgtgatttattgtcgtgtcagaacgctggctggcaaggagtgacgcttaataccacgcgggggcatttctatcgcgcggcgctggaagggttaactgcgcaattacagcgcaatctacagatgctggaaaaaatcgggcactttaaggcctctgaattattgttagtcggtggaggaagtcgcaacacattgtggaatcagattaaagccaatatgcttgatattccggtaaaagttctcgacgacgccgaaacgaccgtcgcaggagctgcgctgttcggttggtatggcgtaggggaatttaacagcccggaagaagcccgcgcacagattcattatcagtaccgttatttctacccgcaaactgaacctgaatttatagaggaagtgtga

SEQ ID NO: 4 - DNA, 1419 bp, Escherichia coli; identical to SEQ ID NO: 3
  source 1..1419: mol_type = genomic DNA; note = fuculose-1-phosphate aldolase(fucA)

SEQ ID NO: 5 - DNA, 1731 bp, Saccharomyces cerevisiae
  source 1..1731: mol_type = genomic DNA; note = hexose transporter(HXT4)
atgtctgaagaagctgcctatcaagaggatacagcagtccaaaatactccagctgatgctttgtcgccagttgaatccgattctaattccgctttgtctactccatccaacaaagctgaaagagatgacatgaaagatttcgacgagaatcacgaagaatctaataactacgttgaaattccaaagaagcccgcctccgcctacgttacagtttccatctgttgtttaatggttgctttcggtggttttgttttcggttgggatactggtaccatttctggttttgttgctcaaactgattttatcagaagatttggtatgaagcaccacgatggtacttattatttgtctaaggttagaactggtttaattgtctccattttcaacattggttgtgccattggtggtattattttagcaaaattaggtgatatgtatggtcgtaaaatgggtttgattgtcgttgttgtcatctacattatcggtatcattatccaaattgcctcaatcaacaagtggtaccaatatttcattggtagaattatctctggtttaggtgtcggtggtattgccgttttatctcctatgttgatttctgaagtctctccaaagcatattagaggtactttggtttcatgttaccaacttatgattactttgggtattttcttgggttactgtacaaactacggtaccaagacctacaccaattctgtccaatggagagttccattaggtctaggtttcgcttgggctttgtttatgattggtggtatgacattcgttccagaatctccacgttatttagttgaagtcggtaaaattgaagaagctaagcgttctattgctctttcaaataaggtcagcgcagacgatccagctgttatggctgaagtcgaagttgttcaagctacagttgaagctgaaaaattagctggtaatgcctcctggggtgaaatatttagcactaagactaaggttttccaacgtttgatcatgggtgctatgattcaatcattgcaacaattgacaggtgataactatttcttctattacggtactaccgttttcactgctgtcggtttggaagattcttttgaaacttctattgtcttgggtattgtcaactttgcttccacctttgttggtattttcttagtcgaaagatatggtcgtcgtagatgtttattatggggtgctgcttccatgacagcttgtatggttgttttcgcttctgttggtgttacaagattgtggccaaatggtaagaagaacgggtcttctaagggtgctggtaactgtatgattgtcttcacatgtttctacttattctgttttgccactacctgggctccaattccatttgttgttaactctgaaactttcccattgagagttaagtccaagtgtatggctattgctcaagcttgtaactggatctggggtttcttgattggtttctttactccatttatttcaggtgctattgatttctactacggttatgttttcatgggctgtttggtcttttcttacttctacgtcttcttcttcgttccagaaactaaaggtttgactttagaagaagttaacaccttatgggaagaaggtgttttgccatggaaatcaccttcttgggttccaccaaacaagagaggtactgactacaacgctgatgatctaatgcatgatgatcaaccattttacaagaagatgttcggaaaaaagtag

SEQ ID NO: 6 - protein, 140 aa, Escherichia coli
  source 1..140: note = fucose mutarotase
MLKTISPLISPELLKVLAEMGHGDEIIFSDAHFPAHSMGPQVIRADGLLVSDLLQAIIPLFELDSYAPPLVMMAAVEGDTLDPEVERRYRNALSLQAPCPDIIRINRFAFYERAQKAFAIVITGERAKYGNILLKKGVTP

SEQ ID NO: 7 - protein, 591 aa, Escherichia coli
  source 1..591: note = fucose isomerase
MKKISLPKIGIRPVIDGRRMGVRESLEEQTMNMAKATAALLTEKLRHACGAAVECVISDTCIAGMAEAAACEEKFSSQNVGLTITVTPCWCYGSETIDMDPTRPKAIWGFNGTERPGAVYLAAALAAHSQKGIPAFSIYGHDVQDADDTSIPADVEEKLLRFARAGLAVASMKGKSYLSLGGVSMGIAGSIVDHNFFESWLGMKVQAVDMTELRRRIDQKIYDEAELEMALAWADKNFRYGEDENNKQYQRNAEQSRAVLRESLLMAMCIRDMMQGNSKLADIGRVEESLGYNAIAAGFQGQRHWTDQYPNGDTAEAILNSSFDWNGVREPFVVATENDSLNGVAMLMGHQLTGTAQVFADVRTYWSPEAIERVTGHKLDGLAEHGIIHLINSGSAALDGSCKQRDSEGNPTMKPHWEISQQEADACLAATEWCPAIHEYFRGGGYSSRFLTEGGVPFTMTRVNIIKGLGPVLQIAEGWSVELPKDVHDILNKRTNSTWPTTWFAPRLTGKGPFTDVYSVMANWGANHGVLTIGHVGADFITLASMLRIPVCMHNVEETKVYRPSAWAAHGMDIEGQDYRACQNYGPLYKR

SEQ ID NO: 8 - protein, 472 aa, Escherichia coli
  source 1..472: note = fuculose kinase
MKQEVILVLDCGATNVRAIAVNRQGKIVARASTPNASDIAMENNTWHQWSLDAILQRFADCCRQINSELTECHIRGIAVTTFGVDGALVDKQGNLLYPIISWKCPRTAAVMDNIERLISAQRLQAISGVGAFSFNTLYKLVWLKENHPQLLERAHAWLFISSLINHRLTGEFTTDITMAGTSQMLDIQQRDFSPQILQATGIPRRLFPRLVEAGEQIGTLQNSAAAMLGLPVGIPVISAGHDTQFALFGAGAEQNEPVLSSGTWEILMVRSAQVDTSLLSQYAGSTCELDSQAGLYNPGMQWLASGVLEWVRKLFWTAETPWQMLIEEARLIAPGADGVKMQCDLLSCQNAGWQGVTLNTTRGHFYRAALEGLTAQLQRNLQMLEKIGHFKASELLLVGGGSRNTLWNQIKANMLDIPVKVLDDAETTVAGAALFGWYGVGEFNSPEEARAQIHYQYRYFYPQTEPEFIEEV

SEQ ID NO: 9 - protein, 472 aa, Escherichia coli; identical to SEQ ID NO: 8
  source 1..472: note = fuculose-1-phosphate aldolase

SEQ ID NO: 10 - protein, 576 aa, Saccharomyces cerevisiae
  source 1..576: note = hexose transporter
MSEEAAYQEDTAVQNTPADALSPVESDSNSALSTPSNKAERDDMKDFDENHEESNNYVEIPKKPASAYVTVSICCLMVAFGGFVFGWDTGTISGFVAQTDFIRRFGMKHHDGTYYLSKVRTGLIVSIFNIGCAIGGIILAKLGDMYGRKMGLIVVVVIYIIGIIIQIASINKWYQYFIGRIISGLGVGGIAVLSPMLISEVSPKHIRGTLVSCYQLMITLGIFLGYCTNYGTKTYTNSVQWRVPLGLGFAWALFMIGGMTFVPESPRYLVEVGKIEEAKRSIALSNKVSADDPAVMAEVEVVQATVEAEKLAGNASWGEIFSTKTKVFQRLIMGAMIQSLQQLTGDNYFFYYGTTVFTAVGLEDSFETSIVLGIVNFASTFVGIFLVERYGRRRCLLWGAASMTACMVVFASVGVTRLWPNGKKNGSSKGAGNCMIVFTCFYLFCFATTWAPIPFVVNSETFPLRVKSKCMAIAQACNWIWGFLIGFFTPFISGAIDFYYGYVFMGCLVFSYFYVFFFVPETKGLTLEEVNTLWEEGVLPWKSPSWVPPNKRGTDYNADDLMHDDQPFYKKMFGKK

SEQ ID NO: 11 - DNA, 28 bp, synthetic construct
  source 1..28: mol_type = other DNA; note = fucA foward primer 1
actagtatggaacgaaataaacttgctc

SEQ ID NO: 12 - DNA, 26 bp, synthetic construct
  source 1..26: mol_type = other DNA; note = fucA reverse primer 2
ctcgagttactcttcaattcgtaacc

SEQ ID NO: 13 - DNA, 29 bp, synthetic construct
  source 1..29: mol_type = other DNA; note = fucI foward primer 1
actagtatgaaaaaaatcagcttaccgaa

SEQ ID NO: 14 - DNA, 26 bp, synthetic construct
  source 1..26: mol_type = other DNA; note = fucI reverse primer 2
ctcgagttaacgcttgtacaacggac

SEQ ID NO: 15 - DNA, 26 bp, synthetic construct
  source 1..26: mol_type = other DNA; note = fucU foward primer 1
actagtatgctgaaaacaatttcgcc

SEQ ID NO: 16 - DNA, 26 bp, synthetic construct
  source 1..26: mol_type = other DNA; note = fucU reverse primer 2
ctcgagttacggtgttaccccttttt

SEQ ID NO: 17 - DNA, 28 bp, synthetic construct
  source 1..28: mol_type = other DNA; note = fucK foward primer 1
actagtatgaaacaagaagttatcctgg

SEQ ID NO: 18 - DNA, 26 bp, synthetic construct
  source 1..26: mol_type = other DNA; note = fucK reverse primer 2
ctcgagtcacacttcctctataaatt

SEQ ID NO: 19 - DNA, 30 bp, synthetic construct
  source 1..30: mol_type = other DNA; note = HXT4 foward primer 1
actagtatgtctgaagaagctgcctatcaa

SEQ ID NO: 20 - DNA, 26 bp, synthetic construct
  source 1..26: mol_type = other DNA; note = HXT4 reverse primer 2
ctcgagctacttttttccgaacatct

SEQ ID NO: 21 - DNA, 8436 bp, synthetic construct
  source 1..8436: mol_type = other DNA; note = prs423GPD_fucA (pRS423GPD harboring fucA from E. coli K12 MG1655)
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctctccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaatctagaactagtatgaaaaaaatcagcttaccgaaaattggtatccgcccggttattgacggtcgtcgcatgggtgttcgtgagtcgcttgaagaacaaacaatgaatatggcgaaagctacggccgcactgctgaccgagaaactgcgccatgcctgcggagctgccgtcgagtgtgtcatttccgatacctgtatcgcgggtatggctgaagccgctgcttgcgaagaaaaattcagcagtcagaatgtaggcctcaccattacggtaacgccttgctggtgctatggcagtgaaaccatcgacatggatccaacccgcccgaaggccatttggggctttaacggcactgaacgccccggcgctgtttacctggcagcggctctggcagctcacagccagaaaggcatcccagcattctccatttacggtcatgacgttcaggatgccgatgacacatcgattcctgccgatgttgaagaaaaactgctgcgctttgcccgcgccggtttggccgtcgccagcatgaaaggtaaaagctatctgtcgctgggcggcgtttcgatgggtatcgccggttccattgttgatcacaacttctttgaatcctggctgggaatgaaagtccaggcggtggatatgaccgaactgcgtcgccgtatcgatcagaagatttacgacgaagccgaattggaaatggcactggcctgggctgataaaaacttccgctatggcgaagatgaaaataacaaacagtatcaacgtaatgccgagcaaagccgcgcagttctgcgcgaaagtttactgatggcgatgtgtatccgcgacatgatgcaaggcaacagcaaactggccgatattggtcgcgtggaagaatcacttggctacaacgccatcgctgcgggcttccaggggcaacgtcactggaccgatcaatatcccaatggtgacaccgccgaagcgatcctcaacagttcatttgactggaatggcgtgcgcgaaccctttgtcgtggcgaccgaaaacgacagtcttaacggcgtggcaatgctaatgggtcaccagctcaccggcaccgctcaggtatttgccgatgtgcgtacctactggtcaccagaagcaattgagcgtgtaacggggcataaactggatggactggcagaacacggcatcatccatttgatcaactccggttctgctgcgctggacggttcctgtaaacaacgcgacagcgaaggtaacccgacgatgaagccacactgggaaatctctcagcaagaggctgacgcttgcctcgccgctaccgaatggtgcccggcgatccacgaatacttccgtggcggcggttactcttcccgcttccttaccgaaggcggcgtcccgttcaccatgactcgtgtcaacatcatcaaaggcctgggaccggtactgcaaatcgcggaaggctggagcgtggaattgccgaaggatgtgcatgacatcctcaacaaacgcaccaactcaacctggccaaccacctggtttgcaccgcgcctcaccggtaaagggccgtttacggatgtgtactcggtaatggcgaactggggcgctaaccatggggttctgaccatcggccacgttggcgcagactttatcactctcgcctccatgctgcgtatcccggtatgtatgcacaacgttgaagagaccaaagtgtatcgtccttctgcctgggctgcgcacggcatggatattgaaggccaggattaccgcgcttgccagaactacggtccgttgtacaagcgttaactcgagacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcgccagtagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacgggaatttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 22 - DNA, 8519 bp, synthetic construct
  source 1..8519: mol_type = other DNA; note = prs424GPD_fucIfucU: pRS424GPD harboring fucI and fucU from E. coli K12 MG1655
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctctccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaatctagaactagtatggaacgaaataaacttgctcgtcagattattgacacttgcctggaaatgacccgcctgggactgaaccaggggacagcggggaacgtcagtgtacgttatcaggatgggatgctgattacgcctacaggcattccatatgaaaaactgacggagtcgcatattgtctttattgatggcaacggtaaacatgaggaaggaaagctcccctcaagcgaatggcgtttccatatggcagcctatcaaagcagaccggatgccaacgcggttgttcacaatcatgccgttcattgcacggcagtttccattcttaaccgatcgatccccgctattcactacatgattgcggcggctggcggtaattctattccttgcgcgccttatgcgacctttggaacacgcgaactttctgaacatgttgcgctggctctcaaaaatcgtaaggcaactttgttacaacatcatgggcttatcgcttgtgaggtgaatctggaaaaagcgttatggctggcgcatgaagttgaagtgctggcgcaactttacctgacgaccctggcgattacggacccggtgccagtgctgagcgatgaagagattgccgtagtgctggagaaattcaaaacctatgggttacgaattgaagagtaactcgagacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgggtaccgagctctccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaatctagaactaatgctgaaaacaatttcgccgttaatttctcccgaactattgaaagtgctggcagagatgggacatggagatgaaattattttttccgatgctcactttcccgcccattcgatgggaccgcaggtgatccgcgctgatggcctgttggtgagcgacttgctccaggcgattatcccgttatttgaactggacagttatgcaccgccgctggtgatgatggcggcggtagaaggtgacactctcgatcctgaagtagaacgacgttaccgtaatgcgctttcactacaagccccgtgtcctgacatcatccgcatcaatcgttttgcgttttatgaacgggcgcaaaaagcctttgcgatcgttatcacaggcgaacgagcgaagtacgggaatattcttttaaaaaaaggggtaacaccgtaagtctcgagacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcataggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgtctattaaatgcttcctatattatatatatagtaatgtcgtttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 23 - DNA, 8007 bp, synthetic construct
  source 1..8007: mol_type = other DNA; note = prs426GPD_fucK: pRS426GPD harboring fucK from E. coli K12 MG1655
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctctccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaatctagaactagtatgaaacaagaagttatcctggtactcgactgtggcgcgaccaatgtcagggccatcgcggttaatcggcagggcaaaattgttgcccgcgcctcaacgcctaatgccagcgatatcgcgatggaaaacaacacctggcaccagtggtctttagacgccattttgcaacgctttgctgattgctgtcggcaaatcaatagtgaactgactgaatgccacatccgcggtatcgccgtcaccacctttggtgtggatggcgctctggtagataagcaaggcaatctgctctatccgattattagctggaaatgtccgcgaacagcagcggttatggacaatattgaacggttaatctccgcacagcggttgcaggctatttctggcgtcggagcctttagtttcaatacgttatataagttggtgtggttgaaagaaaatcatccacaactgctggaacgcgcgcacgcctggctctttatttcgtcgctgattaaccaccgtttaaccggcgaattcactactgatatcacgatggccggaaccagccagatgctggatatccagcaacgcgatttcagtccgcaaattttacaagccaccggtattccacgccgactcttccctcgtctggtggaagcgggtgaacagattggtacgctacagaacagcgccgcagcaatgctcggcttacccgttggcataccggtgatttccgcaggtcacgatacccagttcgccctttttggcgctggtgctgaacaaaatgaacccgtgctctcttccggtacatgggaaattttaatggttcgcagcgcccaggttgatacttcgctgttaagtcagtacgccggttccacctgcgaactggatagccaggcagggttgtataacccaggtatgcaatggctggcatccggcgtgctggaatgggtgagaaaactgttctggacggctgaaacaccctggcaaatgttgattgaagaagctcgtctgatcgcgcctggcgcggatggcgtaaaaatgcagtgtgatttattgtcgtgtcagaacgctggctggcaaggagtgacgcttaataccacgcgggggcatttctatcgcgcggcgctggaagggttaactgcgcaattacagcgcaatctacagatgctggaaaaaatcgggcactttaaggcctctgaattattgttagtcggtggaggaagtcgcaacacattgtggaatcagattaaagccaatatgcttgatattccggtaaaagttctcgacgacgccgaaacgaccgtcgcaggagctgcgctgttcggttggtatggcgtaggggaatttaacagcccggaagaagcccgcgcacagattcattatcagtaccgttatttctacccgcaaactgaacctgaatttatagaggaagtgtgactcgagacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 24 - DNA, 10708 bp, synthetic construct
  source 1..10708: mol_type = other DNA; note = prs426GPD_fucKHXT4: pRS426GPD harboring fucK from E. coli K12 MG1655 and HXT4 from S. cerevisiae CEN.PK2-1D
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctctccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaatctagaactagtatgaaacaagaagttatcctggtactcgactgtggcgcgaccaatgtcagggccatcgcggttaatcggcagggcaaaattgttgcccgcgcctcaacgcctaatgccagcgatatcgcgatggaaaacaacacctggcaccagtggtctttagacgccattttgcaacgctttgctgattgctgtcggcaaatcaatagtgaactgactgaatgccacatccgcggtatcgccgtcaccacctttggtgtggatggcgctctggtagataagcaaggcaatctgctctatccgattattagctggaaatgtccgcgaacagcagcggttatggacaatattgaacggttaatctccgcacagcggttgcaggctatttctggcgtcggagcctttagtttcaatacgttatataagttggtgtggttgaaagaaaatcatccacaactgctggaacgcgcgcacgcctggctctttatttcgtcgctgattaaccaccgtttaaccggcgaattcactactgatatcacgatggccggaaccagccagatgctggatatccagcaacgcgatttcagtccgcaaattttacaagccaccggtattccacgccgactcttccctcgtctggtggaagcgggtgaacagattggtacgctacagaacagcgccgcagcaatgctcggcttacccgttggcataccggtgatttccgcaggtcacgatacccagttcgccctttttggcgctggtgctgaacaaaatgaacccgtgctctcttccggtacatgggaaattttaatggttcgcagcgcccaggttgatacttcgctgttaagtcagtacgccggttccacctgcgaactggatagccaggcagggttgtataacccaggtatgcaatggctggcatccggcgtgctggaatgggtgagaaaactgttctggacggctgaaacaccctggcaaatgttgattgaagaagctcgtctgatcgcgcctggcgcggatggcgtaaaaatgcagtgtgatttattgtcgtgtcagaacgctggctggcaaggagtgacgcttaataccacgcgggggcatttctatcgcgcggcgctggaagggttaactgcgcaattacagcgcaatctacagatgctggaaaaaatcgggcactttaaggcctctgaattattgttagtcggtggaggaagtcgcaacacattgtggaatcagattaaagccaatatgcttgatattccggtaaaagttctcgacgacgccgaaacgaccgtcgcaggagctgcgctgttcggttggtatggcgtaggggaatttaacagcccggaagaagcccgcgcacagattcattatcagtaccgttatttctacccgcaaactgaacctgaatttatagaggaagtgtgactcgagacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgggtaccgagctctccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaatctagaactagtatgtctgaagaagctgcctatcaagaggatacagcagtccaaaatactccagctgatgctttgtcgccagttgaatccgattctaattccgctttgtctactccatccaacaaagctgaaagagatgacatgaaagatttcgacgagaatcacgaagaatctaataactacgttgaaattccaaagaagcccgcctccgcctacgttacagtttccatctgttgtttaatggttgctttcggtggttttgttttcggttgggatactggtaccatttctggttttgttgctcaaactgattttatcagaagatttggtatgaagcaccacgatggtacttattatttgtctaaggttagaactggtttaattgtctccattttcaacattggttgtgccattggtggtattattttagcaaaattaggtgatatgtatggtcgtaaaatgggtttgattgtcgttgttgtcatctacattatcggtatcattatccaaattgcctcaatcaacaagtggtaccaatatttcattggtagaattatctctggtttaggtgtcggtggtattgccgttttatctcctatgttgatttctgaagtctctccaaagcatattagaggtactttggtttcatgttaccaacttatgattactttgggtattttcttgggttactgtacaaactacggtaccaagacctacaccaattctgtccaatggagagttccattaggtctaggtttcgcttgggctttgtttatgattggtggtatgacattcgttccagaatctccacgttatttagttgaagtcggtaaaattgaagaagctaagcgttctattgctctttcaaataaggtcagcgcagacgatccagctgttatggctgaagtcgaagttgttcaagctacagttgaagctgaaaaattagctggtaatgcctcctggggtgaaatatttagcactaagactaaggttttccaacgtttgatcatgggtgctatgattcaatcattgcaacaattgacaggtgataactatttcttctattacggtactaccgttttcactgctgtcggtttggaagattcttttgaaacttctattgtcttgggtattgtcaactttgcttccacctttgttggtattttcttagtcgaaagatatggtcgtcgtagatgtttattatggggtgctgcttccatgacagcttgtatggttgttttcgcttctgttggtgttacaagattgtggccaaatggtaagaagaacgggtcttctaagggtgctggtaactgtatgattgtcttcacatgtttctacttattctgttttgccactacctgggctccaattccatttgttgttaactctgaaactttcccattgagagttaagtccaagtgtatggctattgctcaagcttgtaactggatctggggtttcttgattggtttctttactccatttatttcaggtgctattgatttctactacggttatgttttcatgggctgtttggtcttttcttacttctacgtcttcttcttcgttccagaaactaaaggtttgactttagaagaagttaacaccttatgggaagaaggtgttttgccatggaaatcaccttcttgggttccaccaaacaagagaggtactgactacaacgctgatgatctaatgcatgatgatcaaccattttacaagaagatgttcggaaaaaagtagctcgagacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

(19) United States
(12) Patent Application Publication
(10) Pub. No.: US 2026/0347750 A1
(43) Pub. Date: Nov. 5, 2026

(54) RECOMBINANT SACCHAROMYCES BOULARDII CAPABLE OF L-FUCOSE METABOLISM

(71) Applicant: Biocraft, Jeonbuk-do (KR)

(72) Inventors: Kyoung Heon KIM, Seoul (KR); Jung Yeon KIM, Seoul (KR); Yu-eun CHEONG, Suwon-si (KR)

(73) Assignee: Biocraft, Jeonbuk-do (KR)

(21) Appl. No.: 19/108,832

(22) PCT Filed: Sep. 5, 2023
(86) PCT No.: PCT/KR2023/013219

§ 371 Date: Apr. 11, 2025

(30) Foreign Application Priority Data
Sep. 6, 2022 (KR) 10-2022-0112652

(51) Int. Cl. C12N 1/185; C12N 9/12; C12N 9/88; C12N 9/90; C12N 15/81; C12R 1/85

(52) U.S. Cl. CPC C12N 1/185; C12N 9/1205; C12N 9/88; C12N 9/90; C12N 15/81; C12Y 207/01051; C12Y 401/02017; C12Y 501/03; C12Y 503/01025; C12R 2001/85

(57) ABSTRACT

The present invention relates to recombinant Saccharomyces boulardii) that can metabolize L-fucose, which is an intestinal mucin-derived sugar. The present invention may provide genetically-engineered Saccharomyces boulardii having improved intestinal survival and intestinal metabolic activity, making it possible to further utilize various functionalities possessed not only by probiotics but also by the strain.

### TECHNICAL FIELD

The present invention relates to recombinant Saccharomyces boulardii that can metabolize an intestinal mucin-derived sugar, L-fucose.

### BACKGROUND ART

Saccharomyces boulardii is well known as a probiotic strain that exhibits antibacterial, anti-toxic and immunomodulatory effects in the human intestines. This strain has a similar genome to the same species Saccharomyces cerevisiae, but has a distinct phenotype. As a representative example, due to a point mutation in Pgm2, Saccharomyces cerevisiae metabolizes galactose rapidly whereas Saccharomyces boulardii metabolizes it slowly.

In addition, Saccharomyces boulardii can easily grow at the normal human body temperature of 37 °C, and has the characteristic of adhering to intestinal epithelial cells of humans or mice. In addition, Saccharomyces boulardii is more resistant to environmental stresses such as a high temperature and acidity than Saccharomyces cerevisiae.

Due to these phenotype characteristics, Saccharomyces boulardii has been reported to have higher intestinal metabolic activity than Saccharomyces cerevisiae and to provide various health benefits to humans. Particularly, preventive and therapeutic effects on many gastrointestinal diseases caused by pathogens, such as Escherichia coli, Salmonella enterica serotype Typhimurium, Campylobacter jejuni, and Clostridium difficile, have been reported through numerous clinical trials. This treatment method has been used for decades as a common medicine to treat or prevent diarrhea in Europe, Africa, and the United States.

Generally, Saccharomyces boulardii persists in the human and mouse intestines only after high-dose oral administration and is completely excreted 3 to 7 days after administration. In addition, Saccharomyces boulardii has been reported to colonize intestinal epithelial cells in humans and gnotobiotic mice treated with antibiotics. This suggests that Saccharomyces boulardii can grow in the intestines and exhibits various metabolic activities, but can be easily suppressed by competition with other intestinal microorganisms.

The intestinal microorganisms that have settled in the intestines metabolize molecules secreted by some intestinal epithelial cells. Dietary nutrients provided by the host are competitively consumed by the host and other intestinal microorganisms, so they can be considered an insufficient nutritional supply. To overcome this, some microorganisms metabolize mucins secreted by mammalian hosts. Mucins mainly consist of L-fucose, D-galactose, N-acetogalactosamine, N-acetylglucosamine, D-glucuronic acid, and salicylic acid. Among these, L-fucose is most widely used as a carbon source in intestinal microorganisms.

Microorganisms such as Bacteroides thetaiotaomicron, Bacteroides fragilis, and Bifidobacterium bifidum are known to secrete fucosidase, which cleaves L-fucose at a glycoprotein terminus of a mucin molecule. The separated L-fucose is metabolized through a process of phosphorylation by other microorganisms.

Therefore, the present inventors attempted to improve the intestinal settling ability and survival ability of Saccharomyces boulardii, which has various functionalities, and thus completed a Saccharomyces boulardii strain which is transformed with a recombinant vector including genes expressing enzymes required for L-fucose metabolism so that intestinal microorganisms can metabolize L-fucose, which is most widely used as a carbon source.

### DISCLOSURE

### Technical Problem

The present invention is directed to providing a recombinant Saccharomyces boulardii strain which can metabolize L-fucose.

### Technical Solution

One aspect of the present invention provides a recombinant Saccharomyces boulardii strain that metabolizes L-fucose, which includes a gene encoding fucose mutarotase; a gene encoding fucose isomerase; a gene encoding fuculose kinase; a gene encoding fuculose-1-phosphate aldolase; and a gene encoding a hexose transporter.

In the recombinant Saccharomyces boulardii strain, the specific metabolic process of L-fucose is illustrated in FIG. 1. First, L-fucose is taken into the strain by the hexose transporter. There are two types of L-fucose, α-L-fucose and β-L-fucose, wherein β-L-fucose is converted to α-L-fucose by fucose mutarotase. Next, α-L-fucose is converted to L-fuculose by fucose isomerase (fucI), and L-fuculose is phosphorylated to L-fuculose-1-phosphate by fuculose kinase. Afterward, L-fuculose-1-phosphate is converted to lactaldehyde and dihydroxyacetophenone by the action of fuculose-1-phosphate aldolase. Dihydroxyacetophenone is used to synthesize an intermediate for cell growth, while lactaldehyde is converted to 1,2-propanediol and excreted.

The gene encoding fucose mutarotase; the gene encoding fucose isomerase; the gene encoding fuculose kinase; the gene encoding fuculose-1-phosphate aldolase; and the gene encoding a hexose transporter, which are introduced into the recombinant Saccharomyces boulardii strain of the present invention, may be derived from E. coli.

Specifically, the gene encoding fucose mutarotase may be represented by SEQ ID NO: 1; the gene encoding fucose isomerase may be represented by SEQ ID NO: 2; the gene encoding fuculose kinase may be represented by SEQ ID NO: 3; the gene encoding fuculose-1-phosphate aldolase may be represented by SEQ ID NO: 4; and the gene encoding a hexose transporter may be represented by SEQ ID NO: 5.

The fucose mutarotase may be represented by SEQ ID NO: 6; the fucose isomerase may be represented by SEQ ID NO: 7; the fuculose kinase may be represented by SEQ ID NO: 8; the fuculose-1-phosphate aldolase may be represented by SEQ ID NO: 9; and the hexose transporter may be represented by SEQ ID NO: 10. In addition, the enzymes are proteins which are mutated by one or more of substitution, deletion, translocation, and addition, and have the above enzyme activities are also included in the scope of the enzymes, and include preferably amino acid sequences having at least 80%, 95%, 90%, 93%, 94%, 95%, 96%, 97%, 98%, and 99% sequence identity with the amino acid sequences represented by SEQ ID NOs: 5 to 10.

In the present invention, the fact that a polypeptide has a specific percentage (e.g., 80%, 85%, 90%, 95%, or 99%) of sequence identity to another sequence means that, when the two sequences are aligned, the above percentage of amino acid residues is identical when comparing the sequences. The alignment, and % homology or identity may be determined using any suitable software program known in the art, for example, the content described in the literature [CURRENT PROTOCOLS IN MOLECULAR BIOLOGY (F. M. Ausubel et al. (eds), 1987 Supplement 30 section 7.7.18)]. There are preferable programs, including the GCG Pileup program, FASTA (Pearson et al., 1988 Proc. Natl Acad. Sci USA 85:2444-2448), and BLAST (BLAST Manual, Altschul et al., Natl. Cent. Biotechnol. Inf., Natl Lib. Med. (NCIB NLM NIH), Bethesda, MD, and Altschul et al., 1997 NAR25:3389-3402). Another preferable alignment program is ALIGN Plus (Scientific and Educational Software, PA), preferably using default parameters. Another available sequence software program is the TFASTA Data Searching Program that can be used in the Sequence Software Package Version 6.0 (Genetics Computer Group, University of Wisconsin, Madison, WI).

The recombinant Saccharomyces boulardii strain of the present invention may be prepared by transformation with the recombinant vectors including a gene encoding fucose mutarotase; a gene encoding fucose isomerase; a gene encoding fuculose kinase; a gene encoding fuculose-1-phosphate aldolase; and a gene encoding a hexose transporter.

The vector may be in the form in which the above genes are operably linked. The term “operably linked” used herein may generally mean that a base expression control sequence is operably linked with a base sequence encoding a target protein to perform a function, thereby affecting the expression of the coding base sequence. The operable linkage with the vector may be made using gene recombination technology known in the art, and site-specific DNA cleavage and ligation may be made using cleavage and ligation enzymes known in the art.

The term “vector” refers to any carrier for cloning and/or transfer of bases into host cells. The vector may be a replicon to which another DNA fragment is bound to induce the replication of the bound fragment. “Replicon” refers to any genetic unit (e.g., a plasmid, a phage, a cosmid, a chromosome, or a virus), which functions as a self-DNA replication unit in vivo, that is, can replicate under its own control. The term “vector” may include viral and non-viral carriers for introducing nucleotides into host cells in vitro, ex vivo, or in vivo. The term “vector” may also include mini-spherical DNA. For example, the vector may be a plasmid that does not have a bacterial DNA sequence. The term “vector” may also include a transposon such as Sleeping Beauty (Izsvak et. al. J. Mol. Biol. 302:93-102 (2000)), or an artificial chromosome. Examples of commonly used vectors include native or recombinant plasmids, cosmids and bacteriophages. For example, pWE15, M13, MBL3, MBL4, IXII, ASHII, APII, t10, t11, Charon4A, and Charon21A may be used as phage vectors or cosmid vectors, and plasmid vectors may be used. The vectors that can be used in the present invention are not particularly limited, and any known expression vectors can be used.

According to one embodiment of the present invention, it was confirmed that Candida tropicalis strains into which a gene encoding fucose mutarotase; a gene encoding fucose isomerase; a gene encoding fuculose kinase; a gene encoding fuculose-1-phosphate aldolase; and a gene encoding a hexose transporter were introduced metabolized L-fucose not only to produce an intermediate (biomass) for strain growth, but also to produce and excrete 1,2-propanediol, depending on the survival environment.

### Advantageous Effects

The present invention provides genetically engineered Saccharomyces boulardii that metabolizes L-fucose and has improved intestinal survival and intestinal metabolic activity, thereby enabling further utilization of various functionalities possessed by not only probiotics but also the above strain.

### DESCRIPTION OF DRAWINGS

FIG. 1 shows the results of in silico genome-scale metabolic model analysis of the growth of Saccharomyces cerevisiae during the metabolism of L-fucose: (A) Schematic diagram of fucose metabolism for cell growth and detailed reactions; (B) In silico calculation of fucose uptake and growth rate; and (C) Relationship between oxygen supply and D-glucose or L-fucose growth.

FIG. 2 shows the results of comparing Saccharomyces cerevisiae growth profiles and fucose transport efficiency during the metabolism of L-fucose: (A) Growth profile of wild-type Saccharomyces cerevisiae; (B) Growth profile of Saccharomyces cerevisiae FCT; (C) Intracellular L-fucose concentrations in HXT-Null strain and HXT1-7-overexpressing strain; and (D) Growth profiles of Saccharomyces cerevisiae FCT.

FIG. 3 shows the growth profiles of Saccharomyces boulardii during the metabolism of L-fucose under various aerobic conditions: (A) Growth profile of wild-type Saccharomyces boulardii; (B) Growth profile of Saccharomyces boulardii FCT under aerobic conditions; (C) Growth profile of Saccharomyces boulardii FCT under microaerobic conditions; and (D) Growth profile of Saccharomyces boulardii FCT under anaerobic conditions.

FIG. 4 shows the results of basic flux mode analysis to illustrate fucose metabolism under aerobic and anaerobic conditions: Schematic diagrams of estimated fucose metabolism under (A) aerobic conditions and (B) anaerobic conditions; (C) Amounts of 1,2-PDO and biomass produced by metabolizing fucose under aerobic conditions; and (D) Stoichiometric model for maximum production of 1,2-PDO, biomass, or energy under both conditions.

### MODES OF THE INVENTION

Hereinafter, the present invention will be described in more detail through examples according to the present invention, but the scope of the present invention is not limited by the examples below.

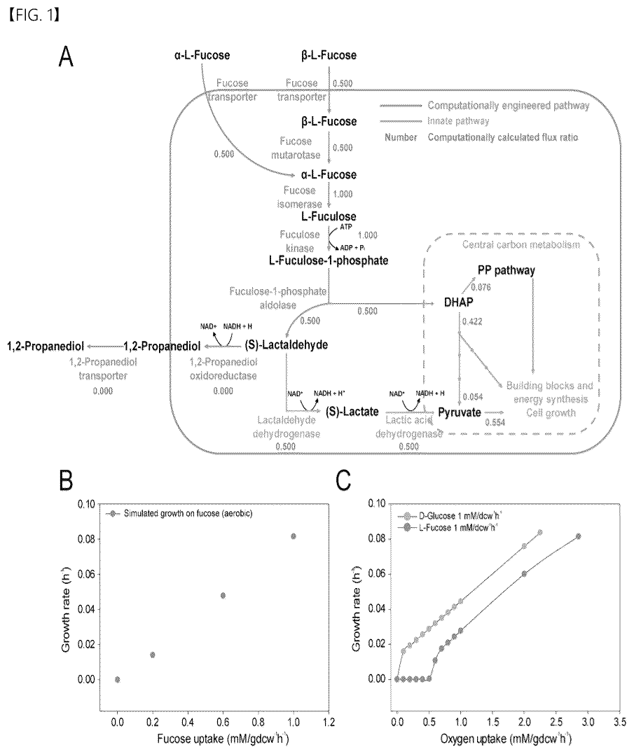

### [Example 1] L-Fucose Metabolism and Cell Growth Simulation of Saccharomyces cerevisiae by Genome-Scale Metabolic Model (GEM) Analysis (FIG. 1)

Microorganisms that metabolize L-fucose are characterized by low energy levels and slow growth rates. To confirm whether yeast can synthesize a sufficient amount of metabolic intermediates for cell growth during the metabolism of L-fucose, an in silico simulation for cell growth was performed using the genome-scale metabolic model (hereinafter, GEM) of Saccharomyces cerevisiae (hereinafter, S. cerevisiae).

Simulation results under aerobic conditions showed that Saccharomyces cerevisiae metabolized L-fucose at a rate of 1 mmol/g dry cell weight (DCW)/h and had a growth rate of 0.08145/h.

Dihydroxyacetophenone, which is an important intermediate in intracellular L-fucose metabolism and an intermediate in the pentose phosphate pathway and glycolysis, is used to synthesize subsequent intermediates. Lactaldehyde, which is another major intermediate, is known to be converted to 1,2-propanediol. However, the growth simulation showed that lactaldehyde is converted to pyruvate by lactaldehyde dehydrogenase and lactate dehydrogenase. In addition, the simulation showed that ethanol is secreted during D-glucose metabolism, but extracellular metabolites such as ethanol, glycerol, and acetate are not secreted during L-fucose metabolism.

Considering the intestinal environment, the oxygen concentration varies depending on the location in the intestines, so the amount of oxygen required to metabolize carbon sources aerobically is an important factor. Therefore, to infer the L-fucose metabolic efficiency of yeast in the intestinal environment, the oxygen demand for the growth of L-fucose-metabolizing yeast was compared with the oxygen demand for the growth of D-glucose-metabolizing yeast. As a result, when 1 mmol of each carbon source is metabolized, yeast metabolizing D-glucose requires less than 0.1 mmol, whereas yeast metabolizing L-fucose requires a minimum of 0.507 mmol of oxygen. These results show that the growth of yeast that can metabolize L-fucose is more oxygen-dependent than the growth of yeast that can metabolize D-glucose.

Under anaerobic conditions, Saccharomyces cerevisiae consumes glucose and efficiently synthesizes metabolizes, but the intermediates produced in L-fucose metabolism are dihydroxyacetophenone and pyruvate. Since these are the initial and final molecules of glycolysis, gluconeogenesis is necessary for the synthesis of an additional intermediate.

Under aerobic conditions, pyruvate is used for energy production via the TCA cycle and dihydroxyacetophenone is used for intermediate synthesis, so the carbon flow in L-fucose metabolism is efficient. However, under anaerobic conditions, the TCA cycle cannot be used, so most of the limited amount of dihydroxyacetophenone is introduced into glycolysis to produce energy, and the metabolic flow for intermediate synthesis becomes slow. In addition, the activation of the (S)-lactaldehyde-(S)-lactate-pyruvate pathway is difficult because of the absence of oxidants. Therefore, it can be concluded that L-fucose metabolism is highly oxygen-dependent due to inefficient carbon flow and a cofactor imbalance.

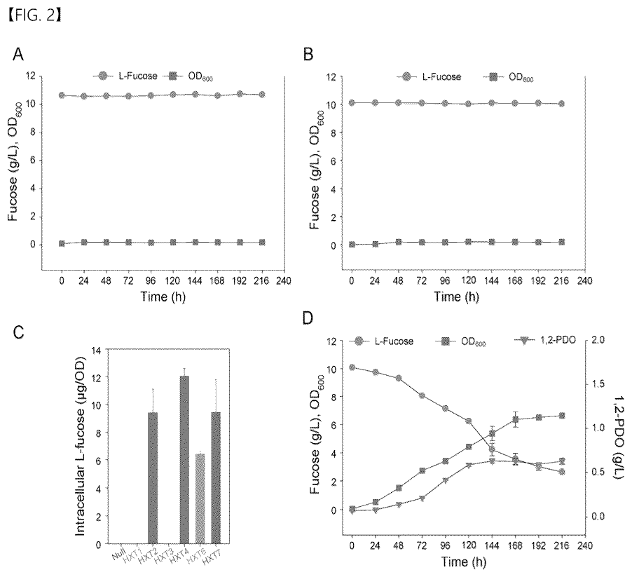

### [Example 2] Development of L-Fucose Metabolic Pathway-Enriched Saccharomyces cerevisiae Strain (FIG. 2)

The GEM results of Example 1 showed that genes related to L-fucose metabolism can be expressed during cell growth of Saccharomyces cerevisiae. In a previous study related to this, it was reported that Saccharomyces cerevisiae can transport a small amount of L-fucose into cells (Yu S, Liu J-J, Yun E J, Kwak S, Kim K H, Jin Y-S. Microb Cell Fact. 2018; 17:101.). In addition, L-fucose permease derived from prokaryotic E. coli is unlikely to be expressed in eukaryotic Saccharomyces cerevisiae. Therefore, the following primer sets were constructed using E. coli K12 MG1655 genomic DNA as a template to obtain the sequences of the gene encoding fucose mutarotase (fucU), the gene encoding fucose isomerase (fucI), the gene encoding fuculose kinase (fucK), and the gene encoding fuculose-1-phosphate aldolase (fucA), excluding fucose permease.

1) fucA
Forward primer 1 (SEQ ID NO: 11):
5′-ACTAGTATGGAACGAAATAAACTTGCTC-3′
Reverse primer 2 (SEQ ID NO: 12):
5′-CTCGAGTTACTCTTCAATTCGTAACC-3′
2) fucI
Forward primer 1 (SEQ ID NO: 13):
5′-ACTAGTATGAAAAAAATCAGCTTACCGAA-3′
Reverse primer 2 (SEQ ID NO: 14):
5′-CTCGAGTTAACGCTTGTACAACGGAC-3′
3) fucU
Forward primer 1 (SEQ ID NO: 15):
5′-ACTAGTATGCTGAAAACAATTTCGCC-3′
Reverse primer 2 (SEQ ID NO: 16):
5′-CTCGAGTTACGGTGTTACCCCTTTTT-3′
4) fucK
Forward primer 1 (SEQ ID NO: 17):
5′-ACTAGTATGAAACAAGAAGTTATCCTGG-3′
Reverse primer 2 (SEQ ID NO: 18):
5′-CTCGAGTCACACTTCCTCTATAAATT-3′
5) HXT4 Forward primer 1 (SEQ ID NO: 19):
5′-ACTAGTATGTCTGAAGAAGCTGCCTATCAA-3′
Reverse primer 2 (SEQ ID NO: 20):
5′-CTCGAGCTACTTTTTTCCGAACATCT-3′

Using the above primers, fucU, fucI, fucK, and fucA amplified by PCR were obtained, and cloned into each of pRS423GPD, pRS424GPD, and pRS426GPD vectors by ligation independent cloning (LIC). The vector sequences are as follows.

1) prs423GPD_fucA (pRS423GPD harboring fucA from E. coli K12 MG1655)
SEQ ID NO: 21)
gacgaaagggcctcgtgatacgcctatttttataggttaatgtcatgataataatggtttcttag
tatgatccaatatcaaaggaaatgatagcattgaaggatgagactaatccaattgaggagtggca
gcatatagaacagctaaagggtagtgctgaaggaagcatacgataccccgcatggaatgggataa
tatcacaggaggtactagactacctttcatcctacataaatagacgcatataagtacgcatttaa
gcataaacacgcactatgccgttcttctcatgtatatatatatacaggcaacacgcagatatagg
tgcgacgtgaacagtgagctgtatgtgcgcagctcgcgttgcattttcggaagcgctcgttttcg
gaaacgctttgaagttcctattccgaagttcctattctctagaaagtataggaacttcagagcgc
ttttgaaaaccaaaagcgctctgaagacgcactttcaaaaaaccaaaaacgcaccggactgtaac
gagctactaaaatattgcgaataccgcttccacaaacattgctcaaaagtatctctttgctatat
atctctgtgctatatccctatataacctacccatccacctttcgctccttgaacttgcatctaaa
ctcgacctctacattttttatgtttatctctagtattactctttagacaaaaaaattgtagtaag
aactattcatagagtgaatcgaaaacaatacgaaaatgtaaacatttcctatacgtagtatatag
agacaaaatagaagaaaccgttcataattttctgaccaatgaagaatcatcaacgctatcacttt
ctgttcacaaagtatgcgcaatccacatcggtatagaatataatcggggatgcctttatcttgaa
aaaatgcacccgcagcttcgctagtaatcagtaaacgcgggaagtggagtcaggctttttttatg
gaagagaaaatagacaccaaagtagccttcttctaaccttaacggacctacagtgcaaaaagtta
tcaagagactgcattatagagcgcacaaaggagaaaaaaagtaatctaagatgctttgttagaaa
aatagcgctctcgggatgcatttttgtagaacaaaaaagaagtatagattctttgttggtaaaat
agcgctctcgcgttgcatttctgttctgtaaaaatgcagctcagattctttgtttgaaaaattag
cgctctcgcgttgcatttttgttttacaaaaatgaagcacagattcttcgttggtaaaatagcgc
tttcgcgttgcatttctgttctgtaaaaatgcagctcagattctttgtttgaaaaattagcgctc
tcgcgttgcatttttgttctacaaaatgaagcacagatgcttcgttcaggtggcacttttcgggg
aaatgtgcgcggaacccctatttgtttatttttctaaatacattcaaatatgtatccgctcatga
gacaataaccctgataaatgcttcaataatattgaaaaaggaagagtatgagtattcaacatttc
cgtgtcgcccttattcccttttttgcggcattttgccttcctgtttttgctcacccagaaacgct
ggtgaaagtaaaagatgctgaagatcagttgggtgcacgagtgggttacatcgaactggatctca
acagcggtaagatccttgagagttttcgccccgaagaacgttttccaatgatgagcacttttaaa
gttctgctatgtggcgcggtattatcccgtattgacgccgggcaagagcaactcggtcgccgcat
acactattctcagaatgacttggttgagtactcaccagtcacagaaaagcatcttacggatggca
tgacagtaagagaattatgcagtgctgccataaccatgagtgataacactgcggccaacttactt
ctgacaacgatcggaggaccgaaggagctaaccgcttttttgcacaacatgggggatcatgtaac
tcgccttgatcgttgggaaccggagctgaatgaagccataccaaacgacgagcgtgacaccacga
tgcctgtagcaatggcaacaacgttgcgcaaactattaactggcgaactacttactctagcttcc
cggcaacaattaatagactggatggaggcggataaagttgcaggaccacttctgcgctcggccct
tccggctggctggtttattgctgataaatctggagccggtgagcgtgggtctcgcggtatcattg
cagcactggggccagatggtaagccccccgtatcgtagttatctacacgacggggagtcaggcaa
ctatggatgaacgaaatagacagatcgctgagataggtgcctcactgattaagcattggtaactg
tcagaccaagtttactcatatatactttagattgatttaaaacttcatttttaatttaaaaggat
ctaggtgaagatcctttttgataatctcatgaccaaaatcccttaacgtgagttttcgttccact
gagcgtcagaccccgtagaaaagatcaaaggatcttcttgagatcctttttttctgcgcgtaatc
tgctgcttgcaaacaaaaaaaccaccgctaccagcggtggtttgtttgccggatcaagagctacc
aactctttttccgaaggtaactggcttcagcagagcgcagataccaaatactgtccttctagtgt
agccgtagttaggccaccacttcaagaactctgtagcaccgcctacatacctcgctctgctaatc
ctgttaccagtggctgctgccagtggcgataagtcgtgtcttaccgggttggactcaagacgata
gttaccggataaggcgcagcggtcgggctgaacggggggttcgtgcacacagcccagcttggagc
gaacgacctacaccgaactgagatacctacagcgtgagctatgagaaagcgccacgcttcccgaa
gggagaaaggcggacaggtatccggtaagcggcagggtcggaacaggagagcgcacgagggagct
tccagggggaaacgcctggtatctttatagtcctgtcgggtttcgccacctctgacttgagcgtc
gatttttgtgatgctcgtcaggggggcggagcctatggaaaaacgccagcaacgcggccttttta
cggttcctggccttttgctggccttttgctcacatgttctttcctgcgttatcccctgattctgt
ggataaccgtattaccgcctttgagtgagctgataccgctcgccgcagccgaacgaccgagcgca
gcgagtcagtgagcgaggaagcggaagagcgcccaatacgcaaaccgcctctccccgcgcgttgg
ccgattcattaatgcagctggcacgacaggtttcccgactggaaagcgggcagtgagcgcaacgc
aattaatgtgagttacctcactcattaggcaccccaggctttacactttatgcttccggctccta
tgttgtgtggaattgtgagcggataacaatttcacacaggaaacagctatgaccatgattacgcc
aagcgcgcaattaaccctcactaaagggaacaaaagctggagctctccaatttattctatccatt
agttgctgatatgtcccaccagccaacacttgatagtatctactcgccattcacttccagcagcg
ccagtagggttgttgagcttagtaaaaatgtgcgcaccacaagcctacatgactccacgtcacat
gaaaccacaccgtggggccttgttgcgctaggaataggatatgcgacgaagacgcttctgcttag
taaccacaccacattttcagggggtcgatctgcttgcttcctttactgtcacgagcggcccataa
tcgcgctttttttttaaaaggcgcgagacagcaaacaggaagctcgggtttcaaccttcggagtg
gtcgcagatctggagactggatctttacaatacagtaaggcaagccaccatctgcttcttaggtg
catgcgacggtatccacgtgcagaacaacatagtctgaagaagggggggaggagcatgttcattc
tctgtagcagtaagagcttggtgataatgaccaaaactggagtctcgaaatcatataaatagaca
atatattttcacacaatgagatttgtagtacagttctattctctctcttgcataaataagaaatt
catcaagaacttggtttgatatttcaccaacacacacaaaaaacagtacttcactaaatttacac
acaaaacaaatctagaactagtatgaaaaaaatcagcttaccgaaaattggtatccgcccggtta
ttgacggtcgtcgcatgggtgttcgtgagtcgcttgaagaacaaacaatgaatatggcgaaagct
acggccgcactgctgaccgagaaactgcgccatgcctgcggagctgccgtcgagtgtgtcatttc
cgatacctgtatcgcgggtatggctgaagccgctgcttgcgaagaaaaattcagcagtcagaatg
taggcctcaccattacggtaacgccttgctggtgctatggcagtgaaaccatcgacatggatcca
acccgcccgaaggccatttggggctttaacggcactgaacgccccggcgctgtttacctggcagc
ggctctggcagctcacagccagaaaggcatcccagcattctccatttacggtcatgacgttcagg
atgccgatgacacatcgattcctgccgatgttgaagaaaaactgctgcgctttgcccgcgccggt
ttggccgtcgccagcatgaaaggtaaaagctatctgtcgctgggcggcgtttcgatgggtatcgc
cggttccattgttgatcacaacttctttgaatcctggctgggaatgaaagtccaggcggtggata
tgaccgaactgcgtcgccgtatcgatcagaagatttacgacgaagccgaattggaaatggcactg
gcctgggctgataaaaacttccgctatggcgaagatgaaaataacaaacagtatcaacgtaatgc
cgagcaaagccgcgcagttctgcgcgaaagtttactgatggcgatgtgtatccgcgacatgatgc
aaggcaacagcaaactggccgatattggtcgcgtggaagaatcacttggctacaacgccatcgct
gcgggcttccaggggcaacgtcactggaccgatcaatatcccaatggtgacaccgccgaagcgat
cctcaacagttcatttgactggaatggcgtgcgcgaaccctttgtcgtggcgaccgaaaacgaca
gtcttaacggcgtggcaatgctaatgggtcaccagctcaccggcaccgctcaggtatttgccgat
gtgcgtacctactggtcaccagaagcaattgagcgtgtaacggggcataaactggatggactggc
agaacacggcatcatccatttgatcaactccggttctgctgcgctggacggttcctgtaaacaac
gcgacagcgaaggtaacccgacgatgaagccacactgggaaatctctcagcaagaggctgacgct
tgcctcgccgctaccgaatggtgcccggcgatccacgaatacttccgtggcggcggttactcttc
ccgcttccttaccgaaggcggcgtcccgttcaccatgactcgtgtcaacatcatcaaaggcctgg
gaccggtactgcaaatcgcggaaggctggagcgtggaattgccgaaggatgtgcatgacatcctc
aacaaacgcaccaactcaacctggccaaccacctggtttgcaccgcgcctcaccggtaaagggcc
gtttacggatgtgtactcggtaatggcgaactggggcgctaaccatggggttctgaccatcggcc
acgttggcgcagactttatcactctcgcctccatgctgcgtatcccggtatgtatgcacaacgtt
gaagagaccaaagtgtatcgtccttctgcctgggctgcgcacggcatggatattgaaggccagga
ttaccgcgcttgccagaactacggtccgttgtacaagcgttaactcgagacaggccccttttcct
ttgtcgatatcatgtaattagttatgtcacgcttacattcacgccctcctcccacatccgctcta
accgaaaaggaaggagttagacaacctgaagtctaggtccctatttattttttttaatagttatg
ttagtattaagaacgttatttatatttcaaatttttcttttttttctgtacaaacgcgtgtacgc
atgtaacattatactgaaaaccttgcttgagaaggttttgggacgctcgggtacccaattcgccc
tatagtgagtcgtattacgcgcgctcactggccgtcgttttacaacgtcgtgactgggaaaaccc
tggcgttacccaacttaatcgccttgcagcacatccccctttcgccagctggcgtaatagcgaag
aggcccgcaccgatcgcccttcccaacagttgcgcagcctgaatggcgaatggcgcgacgcgccc
tgtagcggcgcattaagcgcggcgggtgtggtggttacgcgcagcgtgaccgctacacttgccag
cgccctagcgcccgctcctttcgctttcttcccttcctttctcgccacgttcgccggctttcccc
gtcaagctctaaatcgggggctccctttagggttccgatttagtgctttacggcacctcgacccc
aaaaaacttgattagggtgatggttcacgtagtgggccatcgccctgatagacggtttttcgccc
tttgacgttggagtccacgttctttaatagtggactcttgttccaaactggaacaacactcaacc
ctatctcggtctattcttttgatttataagggattttgccgatttcggcctattggttaaaaaat
gagctgatttaacaaaaatttaacgcgaattttaacaaaatattaacgtttacaatttcctgatg
cggtattttctccttacgcatctgtgcggtatttcacaccgcatagatccgtcgagttcaagaga
aaaaaaaagaaaaagcaaaaagaaaaaaggaaagcgcgcctcgttcagaatgacacgtatagaat
gatgcattaccttgtcatcttcagtatcatactgttcgtatacatacttactgacattcataggt
atacatatatacacatgtatatatatcgtatgctgcagctttaaataatcggtgtcactacataa
gaacacctttggtggagggaacatcgttggtaccattgggcgaggtggcttctcttatggcaacc
gcaagagccttgaacgcactctcactacggtgatgatcattcttgcctcgcagacaatcaacgtg
gagggtaattctgctagcctctgcaaagctttcaagaaaatgcgggatcatctcgcaagagagat
ctcctactttctccctttgcaaaccaagttcgacaactgcgtacggcctgttcgaaagattacca
ccgctctggaaagtgcctcatccaaaggcgcaaatcctgatccaaacctttttactccacgcgcc
agtagggcctctttaaaagcttgaccgagagcaatcccgcagtcttcagtggtgtgatggtcgtc
tatgtgtaagtcaccaatgcactcaacgattagcgaccagccggaatgcttggccagagcatgta
tcatatggtccagaaaccctatacctgtgtggacgttaatcacttgcgattgtgtggcctgttct
gctactgcttctgcctctttttctgggaagatcgagtgctctatcgctaggggaccaccctttaa
agagatcgcaatctgaatcttggtttcatttgtaatacgctttactagggctttctgctctgtca
tctttgccttcgtttatcttgcctgctcattttttagtatattcttcgaagaaatcacattactt
tatataatgtataattcattatgtgataatgccaatcgctaagaaaaaaaaagagtcatccgcta
ggggaaaaaaaaaaatgaaaatcattaccgaggcataaaaaaatatagagtgtactagaggaggc
caagagtaatagaaaaagaaaattgcgggaaaggactgtgttatgacttccctgactaatgccgt
gttcaaacgatacctggcagtgactcctagcgctcaccaagctcttaaaacgggaatttatggtg
cactctcagtacaatctgctctgatgccgcatagttaagccagccccgacacccgccaacacccg
ctgacgcgccctgacgggcttgtctgctcccggcatccgcttacagacaagctgtgaccgtctcc
gggagctgcatgtgtcagaggttttcaccgtcatcaccgaaacgcgcga
2) prs424GPD_fucIfucU: pRS424GPD harboring fucl and fucU from
E. coli K12 MG1655
SEQ ID NO: 22
gacgaaagggcctcgtgatacgcctatttttataggttaatgtcatgataataatggtttcttag
tatgatccaatatcaaaggaaatgatagcattgaaggatgagactaatccaattgaggagtggca
gcatatagaacagctaaagggtagtgctgaaggaagcatacgataccccgcatggaatgggataa
tatcacaggaggtactagactacctttcatcctacataaatagacgcatataagtacgcatttaa
gcataaacacgcactatgccgttcttctcatgtatatatatatacaggcaacacgcagatatagg
tgcgacgtgaacagtgagctgtatgtgcgcagctcgcgttgcattttcggaagcgctcgttttcg
gaaacgctttgaagttcctattccgaagttcctattctctagaaagtataggaacttcagagcgc
ttttgaaaaccaaaagcgctctgaagacgcactttcaaaaaaccaaaaacgcaccggactgtaac
gagctactaaaatattgcgaataccgcttccacaaacattgctcaaaagtatctctttgctatat
atctctgtgctatatccctatataacctacccatccacctttcgctccttgaacttgcatctaaa
ctcgacctctacattttttatgtttatctctagtattactctttagacaaaaaaattgtagtaag
aactattcatagagtgaatcgaaaacaatacgaaaatgtaaacatttcctatacgtagtatatag
agacaaaatagaagaaaccgttcataattttctgaccaatgaagaatcatcaacgctatcacttt
ctgttcacaaagtatgcgcaatccacatcggtatagaatataatcggggatgcctttatcttgaa
aaaatgcacccgcagcttcgctagtaatcagtaaacgcgggaagtggagtcaggctttttttatg
gaagagaaaatagacaccaaagtagccttcttctaaccttaacggacctacagtgcaaaaagtta
tcaagagactgcattatagagcgcacaaaggagaaaaaaagtaatctaagatgctttgttagaaa
aatagcgctctcgggatgcatttttgtagaacaaaaaagaagtatagattctttgttggtaaaat
agcgctctcgcgttgcatttctgttctgtaaaaatgcagctcagattctttgtttgaaaaattag
cgctctcgcgttgcatttttgttttacaaaaatgaagcacagattcttcgttggtaaaatagcgc
tttcgcgttgcatttctgttctgtaaaaatgcagctcagattctttgtttgaaaaattagcgctc
tcgcgttgcatttttgttctacaaaatgaagcacagatgcttcgttcaggtggcacttttcgggg
aaatgtgcgcggaacccctatttgtttatttttctaaatacattcaaatatgtatccgctcatga
gacaataaccctgataaatgcttcaataatattgaaaaaggaagagtatgagtattcaacatttc
cgtgtcgcccttattcccttttttgcggcattttgccttcctgtttttgctcacccagaaacgct
ggtgaaagtaaaagatgctgaagatcagttgggtgcacgagtgggttacatcgaactggatctca
acagcggtaagatccttgagagttttcgccccgaagaacgttttccaatgatgagcacttttaaa
gttctgctatgtggcgcggtattatcccgtattgacgccgggcaagagcaactcggtcgccgcat
acactattctcagaatgacttggttgagtactcaccagtcacagaaaagcatcttacggatggca
tgacagtaagagaattatgcagtgctgccataaccatgagtgataacactgcggccaacttactt
ctgacaacgatcggaggaccgaaggagctaaccgcttttttgcacaacatgggggatcatgtaac
tcgccttgatcgttgggaaccggagctgaatgaagccataccaaacgacgagcgtgacaccacga
tgcctgtagcaatggcaacaacgttgcgcaaactattaactggcgaactacttactctagcttcc
cggcaacaattaatagactggatggaggcggataaagttgcaggaccacttctgcgctcggccct
tccggctggctggtttattgctgataaatctggagccggtgagcgtgggtctcgcggtatcattg
cagcactggggccagatggtaagccccccgtatcgtagttatctacacgacggggagtcaggcaa
ctatggatgaacgaaatagacagatcgctgagataggtgcctcactgattaagcattggtaactg
tcagaccaagtttactcatatatactttagattgatttaaaacttcatttttaatttaaaaggat
ctaggtgaagatcctttttgataatctcatgaccaaaatcccttaacgtgagttttcgttccact
gagcgtcagaccccgtagaaaagatcaaaggatcttcttgagatcctttttttctgcgcgtaatc
tgctgcttgcaaacaaaaaaaccaccgctaccagcggtggtttgtttgccggatcaagagctacc
aactctttttccgaaggtaactggcttcagcagagcgcagataccaaatactgtccttctagtgt
agccgtagttaggccaccacttcaagaactctgtagcaccgcctacatacctcgctctgctaatc
ctgttaccagtggctgctgccagtggcgataagtcgtgtcttaccgggttggactcaagacgata
gttaccggataaggcgcagcggtcgggctgaacggggggttcgtgcacacagcccagcttggagc
gaacgacctacaccgaactgagatacctacagcgtgagctatgagaaagcgccacgcttcccgaa
gggagaaaggcggacaggtatccggtaagcggcagggtcggaacaggagagcgcacgagggagct
tccagggggaaacgcctggtatctttatagtcctgtcgggtttcgccacctctgacttgagcgtc
gatttttgtgatgctcgtcaggggggcggagcctatggaaaaacgccagcaacgcggccttttta
cggttcctggccttttgctggccttttgctcacatgttctttcctgcgttatcccctgattctgt
ggataaccgtattaccgcctttgagtgagctgataccgctcgccgcagccgaacgaccgagcgca
gcgagtcagtgagcgaggaagcggaagagcgcccaatacgcaaaccgcctctccccgcgcgttgg
ccgattcattaatgcagctggcacgacaggtttcccgactggaaagcgggcagtgagcgcaacgc
aattaatgtgagttacctcactcattaggcaccccaggctttacactttatgcttccggctccta
tgttgtgtggaattgtgagcggataacaatttcacacaggaaacagctatgaccatgattacgcc
aagcgcgcaattaaccctcactaaagggaacaaaagctggagctctccaatttattctatccatt
agttgctgatatgtcccaccagccaacacttgatagtatctactcgccattcacttccagcagcg
ccagtagggttgttgagcttagtaaaaatgtgcgcaccacaagcctacatgactccacgtcacat
gaaaccacaccgtggggccttgttgcgctaggaataggatatgcgacgaagacgcttctgcttag
taaccacaccacattttcagggggtcgatctgcttgcttcctttactgtcacgagcggcccataa
tcgcgctttttttttaaaaggcgcgagacagcaaacaggaagctcgggtttcaaccttcggagtg
gtcgcagatctggagactggatctttacaatacagtaaggcaagccaccatctgcttcttaggtg
catgcgacggtatccacgtgcagaacaacatagtctgaagaagggggggaggagcatgttcattc
tctgtagcagtaagagcttggtgataatgaccaaaactggagtctcgaaatcatataaatagaca
atatattttcacacaatgagatttgtagtacagttctattctctctcttgcataaataagaaatt
catcaagaacttggtttgatatttcaccaacacacacaaaaaacagtacttcactaaatttacac
acaaaacaaatctagaactagtatggaacgaaataaacttgctcgtcagattattgacacttgcc
tggaaatgacccgcctgggactgaaccaggggacagcggggaacgtcagtgtacgttatcaggat
gggatgctgattacgcctacaggcattccatatgaaaaactgacggagtcgcatattgtctttat
tgatggcaacggtaaacatgaggaaggaaagctcccctcaagcgaatggcgtttccatatggcag
cctatcaaagcagaccggatgccaacgcggttgttcacaatcatgccgttcattgcacggcagtt
tccattcttaaccgatcgatccccgctattcactacatgattgcggcggctggcggtaattctat
tccttgcgcgccttatgcgacctttggaacacgcgaactttctgaacatgttgcgctggctctca
aaaatcgtaaggcaactttgttacaacatcatgggcttatcgcttgtgaggtgaatctggaaaaa
gcgttatggctggcgcatgaagttgaagtgctggcgcaactttacctgacgaccctggcgattac
ggacccggtgccagtgctgagcgatgaagagattgccgtagtgctggagaaattcaaaacctatg
ggttacgaattgaagagtaactcgagacaggccccttttcctttgtcgatatcatgtaattagtt
atgtcacgcttacattcacgccctcctcccacatccgctctaaccgaaaaggaaggagttagaca
acctgaagtctaggtccctatttattttttttaatagttatgttagtattaagaacgttatttat
atttcaaatttttcttttttttctgtacaaacgcgtgtacgcatgtaacattatactgaaaacct
tgcttgagaaggttttgggacgctcgggtaccgagctctccaatttattctatccattagttgct
gatatgtcccaccagccaacacttgatagtatctactcgccattcacttccagcagcgccagtag
ggttgttgagcttagtaaaaatgtgcgcaccacaagcctacatgactccacgtcacatgaaacca
caccgtggggccttgttgcgctaggaataggatatgcgacgaagacgcttctgcttagtaaccac
accacattttcagggggtcgatctgcttgcttcctttactgtcacgagcggcccataatcgcgct
ttttttttaaaaggcgcgagacagcaaacaggaagctcgggtttcaaccttcggagtggtcgcag
atctggagactggatctttacaatacagtaaggcaagccaccatctgcttcttaggtgcatgcga
cggtatccacgtgcagaacaacatagtctgaagaagggggggaggagcatgttcattctctgtag
cagtaagagcttggtgataatgaccaaaactggagtctcgaaatcatataaatagacaatatatt
ttcacacaatgagatttgtagtacagttctattctctctcttgcataaataagaaattcatcaag
aacttggtttgatatttcaccaacacacacaaaaaacagtacttcactaaatttacacacaaaac
aaatctagaactaatgctgaaaacaatttcgccgttaatttctcccgaactattgaaagtgctgg
cagagatgggacatggagatgaaattattttttccgatgctcactttcccgcccattcgatggga
ccgcaggtgatccgcgctgatggcctgttggtgagcgacttgctccaggcgattatcccgttatt
tgaactggacagttatgcaccgccgctggtgatgatggcggcggtagaaggtgacactctcgatc
ctgaagtagaacgacgttaccgtaatgcgctttcactacaagccccgtgtcctgacatcatccgc
atcaatcgttttgcgttttatgaacgggcgcaaaaagcctttgcgatcgttatcacaggcgaacg
agcgaagtacgggaatattcttttaaaaaaaggggtaacaccgtaagtctcgagacaggcccctt
ttcctttgtcgatatcatgtaattagttatgtcacgcttacattcacgccctcctcccacatccg
ctctaaccgaaaaggaaggagttagacaacctgaagtctaggtccctatttattttttttaatag
ttatgttagtattaagaacgttatttatatttcaaatttttcttttttttctgtacaaacgcgtg
tacgcatgtaacattatactgaaaaccttgcttgagaaggttttgggacgctcgggtacccaatt
cgccctatagtgagtcgtattacgcgcgctcactggccgtcgttttacaacgtcgtgactgggaa
aaccctggcgttacccaacttaatcgccttgcagcacatccccctttcgccagctggcgtaatag
cgaagaggcccgcaccgatcgcccttcccaacagttgcgcagcctgaatggcgaatggcgcgacg
cgccctgtagcggcgcattaagcgcggcgggtgtggtggttacgcgcagcgtgaccgctacactt
gccagcgccctagcgcccgctcctttcgctttcttcccttcctttctcgccacgttcgccggctt
tccccgtcaagctctaaatcgggggctccctttagggttccgatttagtgctttacggcacctcg
accccaaaaaacttgattagggtgatggttcacgtagtgggccatcgccctgatagacggttttt
cgccctttgacgttggagtccacgttctttaatagtggactcttgttccaaactggaacaacact
caaccctatctcggtctattcttttgatttataagggattttgccgatttcggcctattggttaa
aaaatgagctgatttaacaaaaatttaacgcgaattttaacaaaatattaacgtttacaatttcc
tgatgcggtattttctccttacgcatctgtgcggtatttcacaccgcataggcaagtgcacaaac
aatacttaaataaatactactcagtaataacctatttcttagcatttttgacgaaatttgctatt
ttgttagagtcttttacaccatttgtctccacacctccgcttacatcaacaccaataacgccatt
taatctaagcgcatcaccaacattttctggcgtcagtccaccagctaacataaaatgtaagcttt
cggggctctcttgccttccaacccagtcagaaatcgagttccaatccaaaagttcacctgtccca
cctgcttctgaatcaaacaagggaataaacgaatgaggtttctgtgaagctgcactgagtagtat
gttgcagtcttttggaaatacgagtcttttaataactggcaaaccgaggaactcttggtattctt
gccacgactcatctccatgcagttggacgatatcaatgccgtaatcattgaccagagccaaaaca
tcctccttaggttgattacgaaacacgccaaccaagtatttcggagtgcctgaactatttttata
tgcttttacaagacttgaaattttccttgcaataaccgggtcaattgttctctttctattgggca
cacatataatacccagcaagtcagcatcggaatctagagcacattctgcggcctctgtgctctgc
aagccgcaaactttcaccaatggaccagaactacctgtgaaattaataacagacatactccaagc
tgcctttgtgtgcttaatcacgtatactcacgtgctcaatagtcaccaatgccctccctcttggc
cctctccttttcttttttcgaccgaattaattcttaatcggcaaaaaaagaaaagctccggatca
agattgtacgtaaggtgacaagctatttttcaataaagaatatcttccactactgccatctggcg
tcataactgcaaagtacacatatattacgatgctgtctattaaatgcttcctatattatatatat
agtaatgtcgtttatggtgcactctcagtacaatctgctctgatgccgcatagttaagccagccc
cgacacccgccaacacccgctgacgcgccctgacgggcttgtctgctcccggcatccgcttacag
acaagctgtgaccgtctccgggagctgcatgtgtcagaggttttcaccgtcatcaccgaaacgcg
cga
3) prs426GPD_fuck: pRS426GPD harboring fuck from E. coli K12 MG1655
SEQ ID NO: 23
gacgaaagggcctcgtgatacgcctatttttataggttaatgtcatgataataatggtttcttag
tatgatccaatatcaaaggaaatgatagcattgaaggatgagactaatccaattgaggagtggca
gcatatagaacagctaaagggtagtgctgaaggaagcatacgataccccgcatggaatgggataa
tatcacaggaggtactagactacctttcatcctacataaatagacgcatataagtacgcatttaa
gcataaacacgcactatgccgttcttctcatgtatatatatatacaggcaacacgcagatatagg
tgcgacgtgaacagtgagctgtatgtgcgcagctcgcgttgcattttcggaagcgctcgttttcg
gaaacgctttgaagttcctattccgaagttcctattctctagaaagtataggaacttcagagcgc
ttttgaaaaccaaaagcgctctgaagacgcactttcaaaaaaccaaaaacgcaccggactgtaac
gagctactaaaatattgcgaataccgcttccacaaacattgctcaaaagtatctctttgctatat
atctctgtgctatatccctatataacctacccatccacctttcgctccttgaacttgcatctaaa
ctcgacctctacattttttatgtttatctctagtattactctttagacaaaaaaattgtagtaag
aactattcatagagtgaatcgaaaacaatacgaaaatgtaaacatttcctatacgtagtatatag
agacaaaatagaagaaaccgttcataattttctgaccaatgaagaatcatcaacgctatcacttt
ctgttcacaaagtatgcgcaatccacatcggtatagaatataatcggggatgcctttatcttgaa
aaaatgcacccgcagcttcgctagtaatcagtaaacgcgggaagtggagtcaggctttttttatg
gaagagaaaatagacaccaaagtagccttcttctaaccttaacggacctacagtgcaaaaagtta
tcaagagactgcattatagagcgcacaaaggagaaaaaaagtaatctaagatgctttgttagaaa
aatagcgctctcgggatgcatttttgtagaacaaaaaagaagtatagattctttgttggtaaaat
agcgctctcgcgttgcatttctgttctgtaaaaatgcagctcagattctttgtttgaaaaattag
cgctctcgcgttgcatttttgttttacaaaaatgaagcacagattcttcgttggtaaaatagcgc
tttcgcgttgcatttctgttctgtaaaaatgcagctcagattctttgtttgaaaaattagcgctc
tcgcgttgcatttttgttctacaaaatgaagcacagatgcttcgttcaggtggcacttttcgggg
aaatgtgcgcggaacccctatttgtttatttttctaaatacattcaaatatgtatccgctcatga
gacaataaccctgataaatgcttcaataatattgaaaaaggaagagtatgagtattcaacatttc
cgtgtcgcccttattcccttttttgcggcattttgccttcctgtttttgctcacccagaaacgct
ggtgaaagtaaaagatgctgaagatcagttgggtgcacgagtgggttacatcgaactggatctca
acagcggtaagatccttgagagttttcgccccgaagaacgttttccaatgatgagcacttttaaa
gttctgctatgtggcgcggtattatcccgtattgacgccgggcaagagcaactcggtcgccgcat
acactattctcagaatgacttggttgagtactcaccagtcacagaaaagcatcttacggatggca
tgacagtaagagaattatgcagtgctgccataaccatgagtgataacactgcggccaacttactt
ctgacaacgatcggaggaccgaaggagctaaccgcttttttgcacaacatgggggatcatgtaac
tcgccttgatcgttgggaaccggagctgaatgaagccataccaaacgacgagcgtgacaccacga
tgcctgtagcaatggcaacaacgttgcgcaaactattaactggcgaactacttactctagcttcc
cggcaacaattaatagactggatggaggcggataaagttgcaggaccacttctgcgctcggccct
tccggctggctggtttattgctgataaatctggagccggtgagcgtgggtctcgcggtatcattg
cagcactggggccagatggtaagccctcccgtatcgtagttatctacacgacggggagtcaggca
actatggatgaacgaaatagacagatcgctgagataggtgcctcactgattaagcattggtaact
gtcagaccaagtttactcatatatactttagattgatttaaaacttcatttttaatttaaaagga
tctaggtgaagatcctttttgataatctcatgaccaaaatcccttaacgtgagttttcgttccac
tgagcgtcagaccccgtagaaaagatcaaaggatcttcttgagatcctttttttctgcgcgtaat
ctgctgcttgcaaacaaaaaaaccaccgctaccagcggtggtttgtttgccggatcaagagctac
caactctttttccgaaggtaactggcttcagcagagcgcagataccaaatactgtccttctagtg
tagccgtagttaggccaccacttcaagaactctgtagcaccgcctacatacctcgctctgctaat
cctgttaccagtggctgctgccagtggcgataagtcgtgtcttaccgggttggactcaagacgat
agttaccggataaggcgcagcggtcgggctgaacggggggttcgtgcacacagcccagcttggag
cgaacgacctacaccgaactgagatacctacagcgtgagctatgagaaagcgccacgcttcccga
agggagaaaggcggacaggtatccggtaagcggcagggtcggaacaggagagcgcacgagggagc
ttccagggggaaacgcctggtatctttatagtcctgtcgggtttcgccacctctgacttgagcgt
cgatttttgtgatgctcgtcaggggggcggagcctatggaaaaacgccagcaacgcggccttttt
acggttcctggccttttgctggccttttgctcacatgttctttcctgcgttatcccctgattctg
tggataaccgtattaccgcctttgagtgagctgataccgctcgccgcagccgaacgaccgagcgc
agcgagtcagtgagcgaggaagcggaagagcgcccaatacgcaaaccgcctctccccgcgcgttg
gccgattcattaatgcagctggcacgacaggtttcccgactggaaagcgggcagtgagcgcaacg
caattaatgtgagttacctcactcattaggcaccccaggctttacactttatgcttccggctcct
atgttgtgtggaattgtgagcggataacaatttcacacaggaaacagctatgaccatgattacgc
caagcgcgcaattaaccctcactaaagggaacaaaagctggagctctccaatttattctatccat
tagttgctgatatgtcccaccagccaacacttgatagtatctactcgccattcacttccagcagc
gccagtagggttgttgagcttagtaaaaatgtgcgcaccacaagcctacatgactccacgtcaca
tgaaaccacaccgtggggccttgttgcgctaggaataggatatgcgacgaagacgcttctgctta
gtaaccacaccacattttcagggggtcgatctgcttgcttcctttactgtcacgagcggcccata
atcgcgctttttttttaaaaggcgcgagacagcaaacaggaagctcgggtttcaaccttcggagt
ggtcgcagatctggagactggatctttacaatacagtaaggcaagccaccatctgcttcttaggt
gcatgcgacggtatccacgtgcagaacaacatagtctgaagaagggggggaggagcatgttcatt
ctctgtagcagtaagagcttggtgataatgaccaaaactggagtctcgaaatcatataaatagac
aatatattttcacacaatgagatttgtagtacagttctattctctctcttgcataaataagaaat
tcatcaagaacttggtttgatatttcaccaacacacacaaaaaacagtacttcactaaatttaca
cacaaaacaaatctagaactagtatgaaacaagaagttatcctggtactcgactgtggcgcgacc
aatgtcagggccatcgcggttaatcggcagggcaaaattgttgcccgcgcctcaacgcctaatgc
cagcgatatcgcgatggaaaacaacacctggcaccagtggtctttagacgccattttgcaacgct
ttgctgattgctgtcggcaaatcaatagtgaactgactgaatgccacatccgcggtatcgccgtc
accacctttggtgtggatggcgctctggtagataagcaaggcaatctgctctatccgattattag
ctggaaatgtccgcgaacagcagcggttatggacaatattgaacggttaatctccgcacagcggt
tgcaggctatttctggcgtcggagcctttagtttcaatacgttatataagttggtgtggttgaaa
gaaaatcatccacaactgctggaacgcgcgcacgcctggctctttatttcgtcgctgattaacca
ccgtttaaccggcgaattcactactgatatcacgatggccggaaccagccagatgctggatatcc
agcaacgcgatttcagtccgcaaattttacaagccaccggtattccacgccgactcttccctcgt
ctggtggaagcgggtgaacagattggtacgctacagaacagcgccgcagcaatgctcggcttacc
cgttggcataccggtgatttccgcaggtcacgatacccagttcgccctttttggcgctggtgctg
aacaaaatgaacccgtgctctcttccggtacatgggaaattttaatggttcgcagcgcccaggtt
gatacttcgctgttaagtcagtacgccggttccacctgcgaactggatagccaggcagggttgta
taacccaggtatgcaatggctggcatccggcgtgctggaatgggtgagaaaactgttctggacgg
ctgaaacaccctggcaaatgttgattgaagaagctcgtctgatcgcgcctggcgcggatggcgta
aaaatgcagtgtgatttattgtcgtgtcagaacgctggctggcaaggagtgacgcttaataccac
gcgggggcatttctatcgcgcggcgctggaagggttaactgcgcaattacagcgcaatctacaga
tgctggaaaaaatcgggcactttaaggcctctgaattattgttagtcggtggaggaagtcgcaac
acattgtggaatcagattaaagccaatatgcttgatattccggtaaaagttctcgacgacgccga
aacgaccgtcgcaggagctgcgctgttcggttggtatggcgtaggggaatttaacagcccggaag
aagcccgcgcacagattcattatcagtaccgttatttctacccgcaaactgaacctgaatttata
gaggaagtgtgactcgagacaggccccttttcctttgtcgatatcatgtaattagttatgtcacg
cttacattcacgccctcctcccacatccgctctaaccgaaaaggaaggagttagacaacctgaag
tctaggtccctatttattttttttaatagttatgttagtattaagaacgttatttatatttcaaa
tttttcttttttttctgtacaaacgcgtgtacgcatgtaacattatactgaaaaccttgcttgag
aaggttttgggacgctcgggtacccaattcgccctatagtgagtcgtattacgcgcgctcactgg
ccgtcgttttacaacgtcgtgactgggaaaaccctggcgttacccaacttaatcgccttgcagca
catccccctttcgccagctggcgtaatagcgaagaggcccgcaccgatcgcccttcccaacagtt
gcgcagcctgaatggcgaatggcgcgacgcgccctgtagcggcgcattaagcgcggcgggtgtgg
tggttacgcgcagcgtgaccgctacacttgccagcgccctagcgcccgctcctttcgctttcttc
ccttcctttctcgccacgttcgccggctttccccgtcaagctctaaatcgggggctccctttagg
gttccgatttagtgctttacggcacctcgaccccaaaaaacttgattagggtgatggttcacgta
gtgggccatcgccctgatagacggtttttcgccctttgacgttggagtccacgttctttaatagt
ggactcttgttccaaactggaacaacactcaaccctatctcggtctattcttttgatttataagg
gattttgccgatttcggcctattggttaaaaaatgagctgatttaacaaaaatttaacgcgaatt
ttaacaaaatattaacgtttacaatttcctgatgcggtattttctccttacgcatctgtgcggta
tttcacaccgcatagggtaataactgatataattaaattgaagctctaatttgtgagtttagtat
acatgcatttacttataatacagttttttagttttgctggccgcatcttctcaaatatgcttccc
agcctgcttttctgtaacgttcaccctctaccttagcatcccttccctttgcaaatagtcctctt
ccaacaataataatgtcagatcctgtagagaccacatcatccacggttctatactgttgacccaa
tgcgtctcccttgtcatctaaacccacaccgggtgtcataatcaaccaatcgtaaccttcatctc
ttccacccatgtctctttgagcaataaagccgataacaaaatctttgtcgctcttcgcaatgtca
acagtacccttagtatattctccagtagatagggagcccttgcatgacaattctgctaacatcaa
aaggcctctaggttcctttgttacttcttctgccgcctgcttcaaaccgctaacaatacctgggc
ccaccacaccgtgtgcattcgtaatgtctgcccattctgctattctgtatacacccgcagagtac
tgcaatttgactgtattaccaatgtcagcaaattttctgtcttcgaagagtaaaaaattgtactt
ggcggataatgcctttagcggcttaactgtgccctccatggaaaaatcagtcaagatatccacat
gtgtttttagtaaacaaattttgggacctaatgcttcaactaactccagtaattccttggtggta
cgaacatccaatgaagcacacaagtttgtttgcttttcgtgcatgatattaaatagcttggcagc
aacaggactaggatgagtagcagcacgttccttatatgtagctttcgacatgatttatcttcgtt
tcctgcaggtttttgttctgtgcagttgggttaagaatactgggcaatttcatgtttcttcaaca
ctacatatgcgtatatataccaatctaagtctgtgctccttccttcgttcttccttctgttcgga
gattaccgaatcaaaaaaatttcaaagaaaccgaaatcaaaaaaaagaataaaaaaaaaatgatg
aattgaattgaaaagctgtggtatggtgcactctcagtacaatctgctctgatgccgcatagtta
agccagccccgacacccgccaacacccgctgacgcgccctgacgggcttgtctgctcccggcatc
cgcttacagacaagctgtgaccgtctccgggagctgcatgtgtcagaggttttcaccgtcatcac
cgaaacgcgcga
4) prs426GPD_fucKHXT4: pRS426GPD harboring fuck from E. coli K12 MG1655
and HXT4 from S. cerevisiae CEN.PK2-1D
SEQ ID NO: 24
gacgaaagggcctcgtgatacgcctatttttataggttaatgtcatgataataatggtttcttag
tatgatccaatatcaaaggaaatgatagcattgaaggatgagactaatccaattgaggagtggca
gcatatagaacagctaaagggtagtgctgaaggaagcatacgataccccgcatggaatgggataa
tatcacaggaggtactagactacctttcatcctacataaatagacgcatataagtacgcatttaa
gcataaacacgcactatgccgttcttctcatgtatatatatatacaggcaacacgcagatatagg
tgcgacgtgaacagtgagctgtatgtgcgcagctcgcgttgcattttcggaagcgctcgttttcg
gaaacgctttgaagttcctattccgaagttcctattctctagaaagtataggaacttcagagcgc
ttttgaaaaccaaaagcgctctgaagacgcactttcaaaaaaccaaaaacgcaccggactgtaac
gagctactaaaatattgcgaataccgcttccacaaacattgctcaaaagtatctctttgctatat
atctctgtgctatatccctatataacctacccatccacctttcgctccttgaacttgcatctaaa
ctcgacctctacattttttatgtttatctctagtattactctttagacaaaaaaattgtagtaag
aactattcatagagtgaatcgaaaacaatacgaaaatgtaaacatttcctatacgtagtatatag
agacaaaatagaagaaaccgttcataattttctgaccaatgaagaatcatcaacgctatcacttt
ctgttcacaaagtatgcgcaatccacatcggtatagaatataatcggggatgcctttatcttgaa
aaaatgcacccgcagcttcgctagtaatcagtaaacgcgggaagtggagtcaggctttttttatg
gaagagaaaatagacaccaaagtagccttcttctaaccttaacggacctacagtgcaaaaagtta
tcaagagactgcattatagagcgcacaaaggagaaaaaaagtaatctaagatgctttgttagaaa
aatagcgctctcgggatgcatttttgtagaacaaaaaagaagtatagattctttgttggtaaaat
agcgctctcgcgttgcatttctgttctgtaaaaatgcagctcagattctttgtttgaaaaattag
cgctctcgcgttgcatttttgttttacaaaaatgaagcacagattcttcgttggtaaaatagcgc
tttcgcgttgcatttctgttctgtaaaaatgcagctcagattctttgtttgaaaaattagcgctc
tcgcgttgcatttttgttctacaaaatgaagcacagatgcttcgttcaggtggcacttttcgggg
aaatgtgcgcggaacccctatttgtttatttttctaaatacattcaaatatgtatccgctcatga
gacaataaccctgataaatgcttcaataatattgaaaaaggaagagtatgagtattcaacatttc
cgtgtcgcccttattcccttttttgcggcattttgccttcctgtttttgctcacccagaaacgct
ggtgaaagtaaaagatgctgaagatcagttgggtgcacgagtgggttacatcgaactggatctca
acagcggtaagatccttgagagttttcgccccgaagaacgttttccaatgatgagcacttttaaa
gttctgctatgtggcgcggtattatcccgtattgacgccgggcaagagcaactcggtcgccgcat
acactattctcagaatgacttggttgagtactcaccagtcacagaaaagcatcttacggatggca
tgacagtaagagaattatgcagtgctgccataaccatgagtgataacactgcggccaacttactt
ctgacaacgatcggaggaccgaaggagctaaccgcttttttgcacaacatgggggatcatgtaac
tcgccttgatcgttgggaaccggagctgaatgaagccataccaaacgacgagcgtgacaccacga
tgcctgtagcaatggcaacaacgttgcgcaaactattaactggcgaactacttactctagcttcc
cggcaacaattaatagactggatggaggcggataaagttgcaggaccacttctgcgctcggccct
tccggctggctggtttattgctgataaatctggagccggtgagcgtgggtctcgcggtatcattg
cagcactggggccagatggtaagccctcccgtatcgtagttatctacacgacggggagtcaggca
actatggatgaacgaaatagacagatcgctgagataggtgcctcactgattaagcattggtaact
gtcagaccaagtttactcatatatactttagattgatttaaaacttcatttttaatttaaaagga
tctaggtgaagatcctttttgataatctcatgaccaaaatcccttaacgtgagttttcgttccac
tgagcgtcagaccccgtagaaaagatcaaaggatcttcttgagatcctttttttctgcgcgtaat
ctgctgcttgcaaacaaaaaaaccaccgctaccagcggtggtttgtttgccggatcaagagctac
caactctttttccgaaggtaactggcttcagcagagcgcagataccaaatactgtccttctagtg
tagccgtagttaggccaccacttcaagaactctgtagcaccgcctacatacctcgctctgctaat
cctgttaccagtggctgctgccagtggcgataagtcgtgtcttaccgggttggactcaagacgat
agttaccggataaggcgcagcggtcgggctgaacggggggttcgtgcacacagcccagcttggag
cgaacgacctacaccgaactgagatacctacagcgtgagctatgagaaagcgccacgcttcccga
agggagaaaggcggacaggtatccggtaagcggcagggtcggaacaggagagcgcacgagggagc
ttccagggggaaacgcctggtatctttatagtcctgtcgggtttcgccacctctgacttgagcgt
cgatttttgtgatgctcgtcaggggggcggagcctatggaaaaacgccagcaacgcggccttttt
acggttcctggccttttgctggccttttgctcacatgttctttcctgcgttatcccctgattctg
tggataaccgtattaccgcctttgagtgagctgataccgctcgccgcagccgaacgaccgagcgc
agcgagtcagtgagcgaggaagcggaagagcgcccaatacgcaaaccgcctctccccgcgcgttg
gccgattcattaatgcagctggcacgacaggtttcccgactggaaagcgggcagtgagcgcaacg
caattaatgtgagttacctcactcattaggcaccccaggctttacactttatgcttccggctcct
atgttgtgtggaattgtgagcggataacaatttcacacaggaaacagctatgaccatgattacgc
caagcgcgcaattaaccctcactaaagggaacaaaagctggagctctccaatttattctatccat
tagttgctgatatgtcccaccagccaacacttgatagtatctactcgccattcacttccagcagc
gccagtagggttgttgagcttagtaaaaatgtgcgcaccacaagcctacatgactccacgtcaca
tgaaaccacaccgtggggccttgttgcgctaggaataggatatgcgacgaagacgcttctgctta
gtaaccacaccacattttcagggggtcgatctgcttgcttcctttactgtcacgagcggcccata
atcgcgctttttttttaaaaggcgcgagacagcaaacaggaagctcgggtttcaaccttcggagt
ggtcgcagatctggagactggatctttacaatacagtaaggcaagccaccatctgcttcttaggt
gcatgcgacggtatccacgtgcagaacaacatagtctgaagaagggggggaggagcatgttcatt
ctctgtagcagtaagagcttggtgataatgaccaaaactggagtctcgaaatcatataaatagac
aatatattttcacacaatgagatttgtagtacagttctattctctctcttgcataaataagaaat
tcatcaagaacttggtttgatatttcaccaacacacacaaaaaacagtacttcactaaatttaca
cacaaaacaaatctagaactagtatgaaacaagaagttatcctggtactcgactgtggcgcgacc
aatgtcagggccatcgcggttaatcggcagggcaaaattgttgcccgcgcctcaacgcctaatgc
cagcgatatcgcgatggaaaacaacacctggcaccagtggtctttagacgccattttgcaacgct
ttgctgattgctgtcggcaaatcaatagtgaactgactgaatgccacatccgcggtatcgccgtc
accacctttggtgtggatggcgctctggtagataagcaaggcaatctgctctatccgattattag
ctggaaatgtccgcgaacagcagcggttatggacaatattgaacggttaatctccgcacagcggt
tgcaggctatttctggcgtcggagcctttagtttcaatacgttatataagttggtgtggttgaaa
gaaaatcatccacaactgctggaacgcgcgcacgcctggctctttatttcgtcgctgattaacca
ccgtttaaccggcgaattcactactgatatcacgatggccggaaccagccagatgctggatatcc
agcaacgcgatttcagtccgcaaattttacaagccaccggtattccacgccgactcttccctcgt
ctggtggaagcgggtgaacagattggtacgctacagaacagcgccgcagcaatgctcggcttacc
cgttggcataccggtgatttccgcaggtcacgatacccagttcgccctttttggcgctggtgctg
aacaaaatgaacccgtgctctcttccggtacatgggaaattttaatggttcgcagcgcccaggtt
gatacttcgctgttaagtcagtacgccggttccacctgcgaactggatagccaggcagggttgta
taacccaggtatgcaatggctggcatccggcgtgctggaatgggtgagaaaactgttctggacgg
ctgaaacaccctggcaaatgttgattgaagaagctcgtctgatcgcgcctggcgcggatggcgta
aaaatgcagtgtgatttattgtcgtgtcagaacgctggctggcaaggagtgacgcttaataccac
gcgggggcatttctatcgcgcggcgctggaagggttaactgcgcaattacagcgcaatctacaga
tgctggaaaaaatcgggcactttaaggcctctgaattattgttagtcggtggaggaagtcgcaac
acattgtggaatcagattaaagccaatatgcttgatattccggtaaaagttctcgacgacgccga
aacgaccgtcgcaggagctgcgctgttcggttggtatggcgtaggggaatttaacagcccggaag
aagcccgcgcacagattcattatcagtaccgttatttctacccgcaaactgaacctgaatttata
gaggaagtgtgactcgagacaggccccttttcctttgtcgatatcatgtaattagttatgtcacg
cttacattcacgccctcctcccacatccgctctaaccgaaaaggaaggagttagacaacctgaag
tctaggtccctatttattttttttaatagttatgttagtattaagaacgttatttatatttcaaa
tttttcttttttttctgtacaaacgcgtgtacgcatgtaacattatactgaaaaccttgcttgag
aaggttttgggacgctcgggtaccgagctctccaatttattctatccattagttgctgatatgtc
ccaccagccaacacttgatagtatctactcgccattcacttccagcagcgccagtagggttgttg
agcttagtaaaaatgtgcgcaccacaagcctacatgactccacgtcacatgaaaccacaccgtgg
ggccttgttgcgctaggaataggatatgcgacgaagacgcttctgcttagtaaccacaccacatt
ttcagggggtcgatctgcttgcttcctttactgtcacgagcggcccataatcgcgcttttttttt
aaaaggcgcgagacagcaaacaggaagctcgggtttcaaccttcggagtggtcgcagatctggag
actggatctttacaatacagtaaggcaagccaccatctgcttcttaggtgcatgcgacggtatcc
acgtgcagaacaacatagtctgaagaagggggggaggagcatgttcattctctgtagcagtaaga
gcttggtgataatgaccaaaactggagtctcgaaatcatataaatagacaatatattttcacaca
atgagatttgtagtacagttctattctctctcttgcataaataagaaattcatcaagaacttggt
ttgatatttcaccaacacacacaaaaaacagtacttcactaaatttacacacaaaacaaatctag
aactagtatgtctgaagaagctgcctatcaagaggatacagcagtccaaaatactccagctgatg
ctttgtcgccagttgaatccgattctaattccgctttgtctactccatccaacaaagctgaaaga
gatgacatgaaagatttcgacgagaatcacgaagaatctaataactacgttgaaattccaaagaa
gcccgcctccgcctacgttacagtttccatctgttgtttaatggttgctttcggtggttttgttt
tcggttgggatactggtaccatttctggttttgttgctcaaactgattttatcagaagatttggt
atgaagcaccacgatggtacttattatttgtctaaggttagaactggtttaattgtctccatttt
caacattggttgtgccattggtggtattattttagcaaaattaggtgatatgtatggtcgtaaaa
tgggtttgattgtcgttgttgtcatctacattatcggtatcattatccaaattgcctcaatcaac
aagtggtaccaatatttcattggtagaattatctctggtttaggtgtcggtggtattgccgtttt
atctcctatgttgatttctgaagtctctccaaagcatattagaggtactttggtttcatgttacc
aacttatgattactttgggtattttcttgggttactgtacaaactacggtaccaagacctacacc
aattctgtccaatggagagttccattaggtctaggtttcgcttgggctttgtttatgattggtgg
tatgacattcgttccagaatctccacgttatttagttgaagtcggtaaaattgaagaagctaagc
gttctattgctctttcaaataaggtcagcgcagacgatccagctgttatggctgaagtcgaagtt
gttcaagctacagttgaagctgaaaaattagctggtaatgcctcctggggtgaaatatttagcac
taagactaaggttttccaacgtttgatcatgggtgctatgattcaatcattgcaacaattgacag
gtgataactatttcttctattacggtactaccgttttcactgctgtcggtttggaagattctttt
gaaacttctattgtcttgggtattgtcaactttgcttccacctttgttggtattttcttagtcga
aagatatggtcgtcgtagatgtttattatggggtgctgcttccatgacagcttgtatggttgttt
tcgcttctgttggtgttacaagattgtggccaaatggtaagaagaacgggtcttctaagggtgct
ggtaactgtatgattgtcttcacatgtttctacttattctgttttgccactacctgggctccaat
tccatttgttgttaactctgaaactttcccattgagagttaagtccaagtgtatggctattgctc
aagcttgtaactggatctggggtttcttgattggtttctttactccatttatttcaggtgctatt
gatttctactacggttatgttttcatgggctgtttggtcttttcttacttctacgtcttcttctt
cgttccagaaactaaaggtttgactttagaagaagttaacaccttatgggaagaaggtgttttgc
catggaaatcaccttcttgggttccaccaaacaagagaggtactgactacaacgctgatgatcta
atgcatgatgatcaaccattttacaagaagatgttcggaaaaaagtagctcgagacaggcccctt
ttcctttgtcgatatcatgtaattagttatgtcacgcttacattcacgccctcctcccacatccg
ctctaaccgaaaaggaaggagttagacaacctgaagtctaggtccctatttattttttttaatag
ttatgttagtattaagaacgttatttatatttcaaatttttcttttttttctgtacaaacgcgtg
tacgcatgtaacattatactgaaaaccttgcttgagaaggttttgggacgctcgggtacccaatt
cgccctatagtgagtcgtattacgcgcgctcactggccgtcgttttacaacgtcgtgactgggaa
aaccctggcgttacccaacttaatcgccttgcagcacatccccctttcgccagctggcgtaatag
cgaagaggcccgcaccgatcgcccttcccaacagttgcgcagcctgaatggcgaatggcgcgacg
cgccctgtagcggcgcattaagcgcggcgggtgtggtggttacgcgcagcgtgaccgctacactt
gccagcgccctagcgcccgctcctttcgctttcttcccttcctttctcgccacgttcgccggctt
tccccgtcaagctctaaatcgggggctccctttagggttccgatttagtgctttacggcacctcg
accccaaaaaacttgattagggtgatggttcacgtagtgggccatcgccctgatagacggttttt
cgccctttgacgttggagtccacgttctttaatagtggactcttgttccaaactggaacaacact
caaccctatctcggtctattcttttgatttataagggattttgccgatttcggcctattggttaa
aaaatgagctgatttaacaaaaatttaacgcgaattttaacaaaatattaacgtttacaatttcc
tgatgcggtattttctccttacgcatctgtgcggtatttcacaccgcatagggtaataactgata
taattaaattgaagctctaatttgtgagtttagtatacatgcatttacttataatacagtttttt
agttttgctggccgcatcttctcaaatatgcttcccagcctgcttttctgtaacgttcaccctct
accttagcatcccttccctttgcaaatagtcctcttccaacaataataatgtcagatcctgtaga
gaccacatcatccacggttctatactgttgacccaatgcgtctcccttgtcatctaaacccacac
cgggtgtcataatcaaccaatcgtaaccttcatctcttccacccatgtctctttgagcaataaag
ccgataacaaaatctttgtcgctcttcgcaatgtcaacagtacccttagtatattctccagtaga
tagggagcccttgcatgacaattctgctaacatcaaaaggcctctaggttcctttgttacttctt
ctgccgcctgcttcaaaccgctaacaatacctgggcccaccacaccgtgtgcattcgtaatgtct
gcccattctgctattctgtatacacccgcagagtactgcaatttgactgtattaccaatgtcagc
aaattttctgtcttcgaagagtaaaaaattgtacttggcggataatgcctttagcggcttaactg
tgccctccatggaaaaatcagtcaagatatccacatgtgtttttagtaaacaaattttgggacct
aatgcttcaactaactccagtaattccttggtggtacgaacatccaatgaagcacacaagtttgt
ttgcttttcgtgcatgatattaaatagcttggcagcaacaggactaggatgagtagcagcacgtt
ccttatatgtagctttcgacatgatttatcttcgtttcctgcaggtttttgttctgtgcagttgg
gttaagaatactgggcaatttcatgtttcttcaacactacatatgcgtatatataccaatctaag
tctgtgctccttccttcgttcttccttctgttcggagattaccgaatcaaaaaaatttcaaagaa
accgaaatcaaaaaaaagaataaaaaaaaaatgatgaattgaattgaaaagctgtggtatggtgc
actctcagtacaatctgctctgatgccgcatagttaagccagccccgacacccgccaacacccgc
tgacgcgccctgacgggcttgtctgctcccggcatccgcttacagacaagctgtgaccgtctccg
ggagctgcatgtgtcagaggttttcaccgtcatcaccgaaacgcgcga

The recombinant vectors were transformed into an E. coli DH5a strain, which is an E. coli strain for replication. Afterward, each recombinant vector was transformed into S. cerevisiae CEN. PK2-1D by a lithium acetate/single-stranded carrier DNA/polyethylene glycol method. As a result, both wild-type Saccharomyces cerevisiae (S. cerevisiae WT) and S. cerevisiae FCT did not metabolize L-fucose in yeast nitrogen base (YNB) media.

This is because yeast cells cannot import enough L-fucose, requiring the introduction of an appropriate transporter. To identify transporters that can be expressed in eukaryotic S. cerevisiae and are efficient for intracellular transport of L-fucose, an HXT-null Saccharomyces cerevisiae strain expressing each hexose transporter derived from S. cerevisiae CEN. PK2-1D was constructed, and L-fucose transport efficiency was compared. As a result, it was confirmed that the HXT4 transporter had the highest L-fucose transport capacity.

Therefore, a transformed S. cerevisiae FCT strain that overexpresses E. coli-derived L-fucose metabolic genes and S. cerevisiae CEN. PK2-1D-derived HXT4 was constructed.

Under aerobic conditions, S. cerevisiae FCT was confirmed to grow in a YNB medium while metabolizing L-fucose (0.27±0.018 mmol fucose/g DCW/h) (Specific growth rate: 0.034±0.001/h). In addition, 1,2-propanediol was produced as an extracellular metabolite (0.045±0.004 mmol 1,2-PDO/g DCW/h).

According to the GEM results of Example 1, S. cerevisiae showed optimal growth when lactaldehyde was converted to lactate rather than 1,2-propanediol (FIG. 1A). The production of 1,2-propanediol appearing without the overexpression of 1,2-PDO oxidoreductase confirmed the possibility that S. cerevisiae has an unidentified 1,2-propanediol oxidoreductase that is expressed during L-fucose metabolism (FIG. 2).

TABLE 1
Strains and plasmids used in this study

| Strain/plasmid | Description |
|---|---|
| Strains | |
| E. coli DH5α | F 80d, lacZΔM15, endA1, recA1, hsdR17(rK mK), supE44, thi-1, gyrA96, |
| | relA1, Δ(lacZYA-argF)U169 |
| S. cerevisiae | MATaura3-52 leu2-3,112 trp1-289 his3ΔMAL2-8c SUC2 |
| CEN.PK2-1D | |
| S. cerevisiae WT | S. cerevisiae CEN.PK2-1D harboring pRS423GPD, pRS424GPD, |
| | pRS426GPD |
| S. cerevisiae FC | S. cerevisiae CEN.PK2-1D harboring pRS423GPD_fucA, pRS424GPD_fucIU, |
| | pRS426GPD_fucK |
| S. cerevisiae FCT | S. cerevisiae CEN.PK2-1D harboring pRS423GPD_fucA, pRS424GPD_fucIU, |
| | pRS426GPD_fucK_HXT4 |
| S. boulardii (ATCC | MATaura3-52 leu2-3,112 trp1-289 his3ΔMAL2-8c SUC2 |
| MYA-796) | |
| S. boulardii WT | S. boulardii harboring pRS423GPD, pRS424GPD, pRS426GPD |
| S. boulardii FCT | S. boulardii harboring pRS423GPD_fucA, pRS424GPD_fucIU, |
| | pRS426GPD_fucK_HXT4 |
| HXT-null S. cerevisiae | S. cerevisiae D452-2 with a xylose pathway and deletion of HXT1-HXT7 |
| S. cerevisiae HXT1 | Hxt-null S. cerevisiae with overexpression of HXT1 |
| S. cerevisiae HXT2 | Hxt-null S. cerevisiae with overexpression of HXT2 |
| S. cerevisiae HXT3 | Hxt-null S. cerevisiae with overexpression of HXT3 |
| S. cerevisiae HXT4 | Hxt-null S. cerevisiae with overexpression of HXT4 |
| S. cerevisiae HXT6 | Hxt-null S. cerevisiae with overexpression of HXT6 |
| S. cerevisiae HXT7 | Hxt-null S. cerevisiae with overexpression of HXT7 |
| Plasmids | |
| prs423GPD | HIS3, GPD promoter, CYC1 terminator, 2μ origin, and Ampr |
| prs424GPD | LEU2, GPD promoter, CYC1 terminator, 2μ origin, and Ampr |
| prs426GPD | URA3, GPD promoter, CYC1 terminator, 2μ origin, and Ampr |
| prs423GPD_fucA | pRS423GPD harboring fucA from E. coli K12 MG1655 |
| prs424GPD_fucIfucU | pRS424GPD harboring fucI and fucU from E. coli K12 MG1655 |
| prs426GPD_fucKHXT4 | pRS426GPD harboring fucK from E. coli K12 MG1655 and HXT4 from S. |
| | cerevisiae CEN.PK2-1D |
| prs426GPD_fucK | pRS426GPD harboring fucK from E. coli K12 MG1655 |

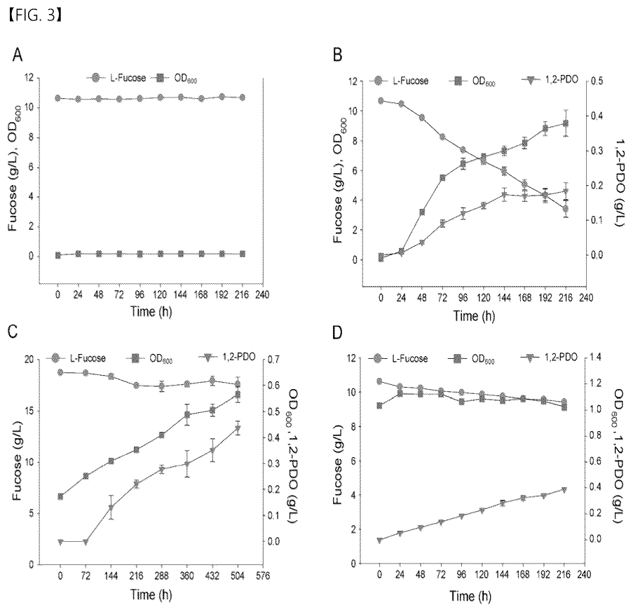

### [Example 3] L-Fucose Metabolism and Fermentation Profiles of Saccharomyces boulardii According to Limited Oxygen Supply (FIG. 3)

To obtain a transformed Saccharomyces boulardii strain that can metabolize L-fucose, the recombinant vector applied to the Saccharomyces cerevisiae FCT strain of Example 2 was cloned into Saccharomyces boulardii, thereby transforming it into a Saccharomyces boulardii FCT strain.

Under anaerobic conditions, it was confirmed that the wild-type Saccharomyces boulardii (Saccharomyces boulardii WT) cannot metabolize L-fucose in a YNB medium, whereas the Saccharomyces boulardii FCT strain can metabolize L-fucose. When L-fucose (0.209±0.004 mmol fucose/g DCW/h) was consumed, it was confirmed that cell growth (growth rate: 0.047±0.001/h) occurred and 1,2-propanediol (0.018±0.003 mmol 1,2-PDO/g DCW/h) was produced.

During the first 72 hours, the Saccharomyces boulardii FCT strain metabolized 14.69±0.98 mmol of L-fucose and produced 1.19±0.16 mmol of 1,2-propanediol. This result shows that, compared to the Saccharomyces cerevisiae FCT strain of Example 1, the Saccharomyces boulardii FCT strain showed higher cell growth and lower 1,2-propanediol production while consuming the same amount of L-fucose.

To investigate the fucose metabolism of the Saccharomyces boulardii FCT strain in an oxygen supply-limited intestinal environment, growth and metabolic profiles were confirmed under microaerobic and anaerobic conditions.

Under microaerobic conditions, the Saccharomyces boulardii FCT strain metabolized 0.081±0.001 mmol fucose/g DCW/h of L-fucose, and showed cell growth (growth rate: 0.002±0.0002/h) and produced 0.050±0.004 mmol of 1,2-propanediol. The result at the first 216 hours showed that the Saccharomyces boulardii FCT strain metabolized 7.70±0.88 mmol of L-fucose and produced 2.89±0.20 mmol of 1,2-PDO.

Under anaerobic conditions, Saccharomyces boulardii metabolized L-fucose (0.045±0.014 mmol fucose/g DCW/h), and produced 0.033±0.003 mmol/g DCW/h of 1,2-propanediol without cell growth. The result at the first 72 hours showed that the Saccharomyces boulardii FCT strain metabolized 3.43±0.27 mmol of L-fucose and produced 1.83±0.07 mmol of 1,2-propanediol. Therefore, when oxygen supply was limited, it was confirmed that only 1,2-propanediol production occurred instead of cell growth.

Since the (S)-lactaldehyde-(S)-lactate-pyruvate pathway is highly oxygen-dependent, when L-fucose was metabolized under anaerobic conditions, the growth of the Saccharomyces boulardii FCT strain was inhibited. The GEM results of Example 1 showed that the key step of L-fucose-dependent yeast growth is the conversion of lactaldehyde to (S)-lactate, which is then converted to pyruvate. In contrast, in the growth profile of the Saccharomyces boulardii FCT strain, it was confirmed that cell growth significantly slowed down under microaerobic conditions and completely stopped under anaerobic conditions. Therefore, it means that the Saccharomyces boulardii FCT strain can metabolize L-fucose. These results suggest that the Saccharomyces boulardii FCT strain can metabolize L-fucose through a specific metabolic pathway different from that predicted by the GEM of Example 1 under limited oxygen supply conditions.

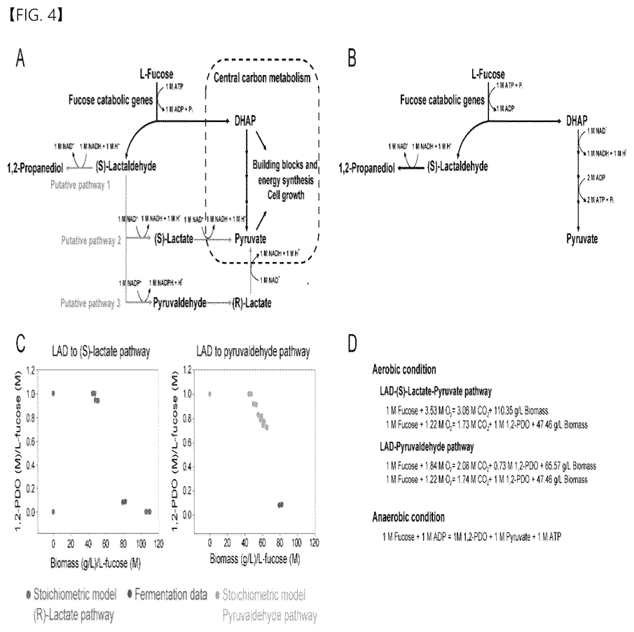

### [Example 4] Prediction of Yeast L-Fucose Metabolism Under Aerobic and Anaerobic Conditions Using Elementary Flux Mode Analysis (EFMA) (FIG. 4)

The GEM results of Example 1 predicted that the growth profile of the Saccharomyces boulardii FCT strain under aerobic conditions would be similar to the fermentation profile, but did not predict the synthesis of 1,2-propanediol. In addition, the results of Example 3 suggested that, even when oxygen supply was limited, the Saccharomyces boulardii FCT strain still metabolized L-fucose, but produced 1,2-propanediol instead of cell growth. Therefore, it can be seen that, in an oxygen-limited environment, the predicted L-fucose metabolic pathway of Example 1 is different from the actual L-fucose metabolic pathway. To determine the cause of these results, the L-fucose metabolic pathway was investigated through elementary flux mode analysis (hereinafter, EFMA).

A series of basic metabolic pathways for cell growth and 1,2-propanediol production were investigated by pathway under aerobic conditions. As a result, it was confirmed that there were 39 models for the conversion pathway from lactaldehyde to (S)-lactate, and 51 models for the conversion pathway from lactaldehyde to pyruvaldehyde. In the conversion pathway from lactaldehyde to (S)-lactate, 1M L-fucose was metabolized to synthesize up to 110.35 g/L of biomass or up to 1M 1,2-propanediol and 47.46 g/L of biomass. It was predicted that the conversion of lactaldehyde to pyruvaldehyde can produce, after metabolizing 1M L-fucose, up to 0.73M 1,2-propanediol and 65.57 g/L of biomass, or up to 1M 1,2-propanediol and 47.46 g/L of biomass. In the conversion pathway model from lactaldehyde to (S)-lactate, all metabolized L-fucose was converted to biomass, which is consistent with the production of 1,2-propanediol and biomass in the fermentation experiment.

Under anaerobic conditions, the EFMA results suggested that the conversion pathway from lactaldehyde to (S)-lactate or the conversion pathway from lactaldehyde to pyruvaldehyde may not be maintained because NAD+ could not be regenerated fast enough. However, it was confirmed that 1M L-fucose could be converted to 1M 1,2-PDO and 1M pyruvate to synthesize 1M ATP. This is consistent with the results of Example 3 that yeast synthesized more 1,2-propanediol than biomass when oxygen is limited. However, the EFMA model for the pyruvaldehyde pathway predicted the synthesis of at least 0.72M 1,2-PDO while metabolizing 1M L-fucose, which is inconsistent with the fermentation data. This result suggests that Saccharomyces boulardii metabolizing L-fucose under aerobic conditions uses the lactaldehyde to (S)-lactate pathway to mostly synthesize biomass along with a small amount of 1,2-PDO. Under microaerobic and anaerobic conditions, the Saccharomyces boulardii FCT strain metabolizes L-fucose and synthesizes more 1,2-PDO with little or no biomass. The EFMA model suggests that the Saccharomyces boulardii FCT strain metabolizes L-fucose and produces 1,2-propanediol, pyruvate and ATP under anaerobic conditions. It was confirmed that, when oxygen is limited, the Saccharomyces boulardii FCT strain can reduce biomass synthesis, but the synthesis of 1,2-propanediol can be increased by enriching the metabolic flow toward the (S)-lactate conversion pathway and 1,2-propanediol and energy production.

1. Recombinant Saccharomyces boulardii that metabolizes L-fucose, comprising:
a gene encoding fucose mutarotase; a gene encoding fucose isomerase; a gene encoding fuculose kinase; a gene encoding fuculose-1-phosphate aldolase; and a gene encoding a hexose transporter.

2. The recombinant Saccharomyces boulardii of claim 1, wherein the gene encoding fucose mutarotase; the gene encoding fucose isomerase; a gene encoding fuculose kinase; the gene encoding fuculose-1-phosphate aldolase; and the gene encoding a hexose transporter are derived from E. coli.

3. The recombinant Saccharomyces boulardii of claim 1, wherein the gene encoding fucose mutarotase is represented by SEQ ID NO: 1; the gene encoding fucose isomerase is represented by SEQ ID NO: 2; the gene encoding fuculose kinase is represented by SEQ ID NO: 3; the gene encoding fuculose-1-phosphate aldolase is represented by SEQ ID NO: 4; and the gene encoding a hexose transporter is represented by SEQ ID NO: 5.

4. The recombinant Saccharomyces boulardii of claim 1, wherein the strain is transformed with a recombinant vector including a gene encoding fucose mutarotase; a gene encoding fucose isomerase; a gene encoding fuculose kinase; a gene encoding fuculose-1-phosphate aldolase; and a gene encoding a hexose transporter.
